(19) United States
(12) Patent Application Publication
(10) Pub. No.: US 2023/0190839 A1
(43) Pub. Date: Jun. 22, 2023

(54) COMPOSITIONS AND COMBINATIONS FOR SUBJECTS SUFFERING FROM ENDOMETRIOSIS

(71) Applicants: GYNOV, BORDEAUX (FR); S.P.M.D., GENTILLY (FR)

(72) Inventor: PIERRE-YVES MOUSSET, LATRESNE (FR)

(21) Appl. No.: 17/925,842

(22) PCT Filed: May 18, 2021
(86) PCT No.: PCT/FR2021/050864

§ 371 Date: Nov. 17, 2022

(30) Foreign Application Priority Data
May 19, 2020 (FR) 2005097

(51) Int. Cl. A61K 36/064; A61P 15/08; A61K 31/05; A61K 36/9066; A61K 31/733; A61K 33/30; A61K 31/121; A61K 36/28; A23L 33/00; A23L 33/105; A23L 33/14; A23L 33/125; A23L 33/15; A23L 33/16; A23L 33/135

(52) U.S. Cl. CPC A61K 36/064; A61P 15/08; A61K 31/05; A61K 36/9066; A61K 31/733; A61K 33/30; A61K 31/121; A61K 36/28; A23L 33/40; A23L 33/105; A23L 33/14; A23L 33/125; A23L 33/15; A23L 33/16; A23L 33/135

(57) ABSTRACT

The present invention relates to the use of a combination of a yeast of the species Saccharomyces cerevisiae, resveratrol and a curcuma extract, for use in the management of a subject who has endometriosis or is susceptible to endometriosis. The combination according to the invention may be used in the treatment or prevention of endometriotic lesions in a subject who has endometriosis or is susceptible to endometriosis, for example to slow the development or to reduce the endometriotic lesions. It may also be used in the treatment or prevention of one or more symptoms or disorders associated with endometriosis. The invention also relates to pharmaceutical or nutraceutical compositions and kits based on said combination.

### TECHNICAL FIELD

The present invention relates to the health field and relates more particularly to the treatment and management of endometriosis as well as symptoms and disorders associated with endometriosis.

### TECHNOLOGICAL BACKGROUND

Endometriosis is defined by the presence of endometrial tissue outside the uterine cavity. This tissue may be present in particular at the level of the walls of the uterus, the fallopian tubes, ovaries, uterosacral ligaments but also at the level of the bladder and colon. Several organs may thus be affected in one and the same patient. In rare cases, endometriotic lesions may also be present at the level of organs located far from the uterus, for example in the lungs. This extrauterine tissue possesses the same characteristics as the endometrium and behaves in the same way under the influence of the ovarian hormones. In this case, the menstrual bleeds have no route out of the body. The problems caused by this trapped blood, which leads to inflammation of the adjacent tissues, are various: lesions, cysts, adhesion between organs. In a subject with endometriosis, we may thus observe superficial nodular lesions on the surface of the ovaries or peritoneum, ovarian cysts (endometriomas), but also deep nodules that invade zones such as the rectovaginal zone, the bladder and the uterosacral ligaments, and adhesions affecting the pelvis, which may lead to a frozen pelvis.

Endometriosis is polymorphic: the organs affected and the associated symptoms and disorders are very variable from one subject to another. A classification exists, allowing endometriosis to be classified in 4 stages as a function of the number of lesions and the organs affected. However, there is no clear correlation between the stage of endometriosis (I-IV) and its effects on fertility and the associated pains.

In fact, the main symptoms and disorders associated with endometriosis are pain and infertility. The major symptom is recurrent pelvic pain, sometimes very acute, in particular at the time of menstruation (dysmenorrhea). This cyclic feature is characteristic of the disease. The lesions are in fact sensitive to the female hormones and behave like uterine tissue. The lesions will therefore proliferate, bleed and leave fibrous scars at each menstrual cycle. In some patients, considerable innervation of the lesions might contribute to the extreme pains sometimes felt. Outside the menstrual period, patients may also suffer at the time of ovulation, during coitus (dyspareunia) or even when they urinate or defecate.

The second symptom is fertility disorders. In fact, a high proportion of patients with endometriosis have hypofertility or infertility.

Moreover, endometriosis is a common pathology, with a considerable socioeconomic impact. This pathology affects at least from 10 to 15% of women. In France, according to an INSEE study (2014), between 2.1 and 4.2 million women are affected by endometriosis, against 3 million for cancers and 2.7 million for type 2 diabetes.

Endometriosis may have very significant effects on patients' lives, in particular because of the very severe pains that it causes, which may lead to stopping work and to absenteeism, and because of infertility and dyspareunia, which have adverse effects on intimate life and family life. The cost of endometriosis in France is estimated at about 9.5 billion euro per year, this figure including both the direct costs (care of patients) but also the indirect costs affecting productivity (sick leaves). The number of hospitalization in connection with endometriosis also increased by +7.5% between 2010 and 2013 (Petit, 2016).

The etiology of endometriosis is still unknown and there is no cure for the disease.

Treatment of endometriosis involves management of pain and infertility, and stabilization of the lesions. In addition to treatment for pain (essentially based on taking nonsteroidal anti-inflammatories (NSAIDs) as and when needed), the first-line treatment is generally hormone treatment intended to suppress menstruation and based on taking a progestogen (of the desogestrel type) or estrogen-progestogen oral contraceptive continuously or on the use of a progestogen implant or coil. This type of treatment may reduce the pains associated with the hormonal response of the endometriotic lesions and may allow stabilization of the lesions, or even a slight decrease in their volume. However, it will not eliminate them completely. Other treatments may be used for establishing artificial menopause, but these treatments are generally only prescribed for a short duration on account of their side-effects. This type of treatment includes the GnRH analogues (leuprolide, triptorelin) and antiandrogens such as danazol, which are generally used for a time of at most 3 to 6 months, for example pre- or post-operatively.

In France, first-line management of infertility is based essentially on medically assisted reproductive technology, in particular on in vitro fertilization.

If medicinal management of pain or infertility fails, or if the nodules become too invasive, the second-line treatment is surgery. However, resection of the lesions associated with endometriosis may be difficult and very invasive, in particular in regions such as the rectosigmoid. Relapses following surgery are also common, in particular because it is impossible to remove all or completely the lesions.

There is therefore a current need for new strategies for the management of patients suffering from endometriosis.

### SUMMARY OF THE INVENTION

The invention relates to a combination of a yeast from Saccharomyces cerevisiae species, resveratrol and a curcuma extract, in particular for use in the management of a subject suffering from endometriosis or susceptible to suffer from endometriosis. The combination according to the invention may be used in the treatment or prevention of endometriotic lesions in a subject suffering from endometriosis or susceptible to endometriosis, for example to slow the development or to reduce endometriotic lesions. The combination according to the invention may also be used in the treatment or prevention of one or more symptoms or disorders associated with endometriosis selected from the group consisting of hypofertility, infertility, pelvic pains in particular dysmenorrhea and dyspareunia, catamenial pains in particular at the urinary or intestinal level, urinary and intestinal disorders, metrorrhagia, proctorrhagia, chronic peritoneal inflammation, intestinal hyperpermeability, chronic fatigue and combinations thereof. The combination according to the invention may in addition be used for normalizing intestinal permeability, modulating the immune state, maintaining fertility and/or decreasing oxidative stress in a subject suffering from endometriosis or susceptible to endometriosis. The yeast from Saccharomyces cerevisiae species, resveratrol and the curcuma extract may be contained in a single pharmaceutical, nutraceutical or nutritional composition or distributed in several separate pharmaceutical, nutraceutical or nutritional compositions intended to be administered simultaneously or sequentially.

In certain embodiments, the subject suffers from endometriosis and has undergone or is to undergo a surgical treatment for endometriotic lesion resection, a medicinal protocol of medically assisted reproduction, in particular in vitro fertilization and/or is under contraceptive treatment.

In other embodiments, the combination according to the invention is included in a food supplement, optionally with one or more ingredients with a physiological or nutritional purpose preferably selected from vitamins, trace elements, minerals, prebiotics, probiotics, antioxidants, amino acids and combinations thereof, and more preferably from folic acid and 5-methyltetrahydrofolic acid, vitamin B6, vitamin B12, zinc, magnesium, calcium, selenium, manganese, inulin, fructo/galacto-oligosaccharides, acacia gum, guar gum, inositol and combinations thereof.

According to an additional aspect, the invention relates to a pharmaceutical, nutraceutical or nutritional composition, preferably in the form of a food supplement, comprising a combination of Saccharomyces cerevisiae yeasts, resveratrol and a curcuma extract, in the following proportions:
from 1 to 50 wt % of yeast Saccharomyces cerevisiae,
from 10 to 50 wt % of resveratrol,
from 1 to 50% by weight for example from 1 to 20% by weight or from 10 to 50% by weight of curcuma extract,

for 100% by weight of the total sum of resveratrol, curcumin and Saccharomyces cerevisiae or for 100% by weight of the total weight of the composition, said composition preferably being suitable for use in the management of subjects suffering from endometriosis or subjects susceptible to endometriosis.

This composition may further comprise one or more prebiotics; and/or vitamins, minerals and/or trace elements; and/or powders or extracts of plants, fruits, vegetables, algae or fungi; and/or one or more probiotics, preferably said probiotic not being a bacterium of the genus Lactobacillus.

The composition according to the invention may be in the form of dosage units, for example in the form of tablets or capsules, each dosage unit comprising:
from 10 to 300 mg, preferably from 25 to 250 mg of resveratrol,
from 1 mg to 300 mg or from 10 to 300 mg, preferably from 5 mg to 25 mg of a curcuma extract or from 25 to 250 mg of a curcuma extract, and
from 1×10⁷ CFU to 1×10¹¹ CFU, preferably 1×10⁸ CFU to 1×10⁹ CFU of Saccharomyces cerevisiae yeast

The composition may further comprise inulin and zinc. For example, the composition according to the invention may be characterized in that it is in the form of dosage units comprising:
from 30 mg to 70 mg of resveratrol, preferably trans-resveratrol,
from 5 to 25 mg of curcuminoids in the form of a curcuma extract associated with a cyclodextrin,
from 1×10⁸ CFU to 1×10¹⁰ CFU, preferably from 2×10⁸ CFU to 6×10⁸ CFU, for example 3×10⁸ CFU to 5×10⁸ CFU of Saccharomyces cerevisiae yeast of the boulardii variety,
optionally from 10 mg to 100 mg, preferably from 25 mg to 50 mg of inulin, and
optionally from 1 mg to 15 mg of zinc (Zn2+) preferably from 2 mg to 8 mg of zinc, for example added in the form of zinc bisglycinate.

The invention also relates to a pharmaceutical, nutraceutical or nutritional kit comprising the combination of two compositions:
a first composition comprising a Saccharomyces cerevisiae yeast preferably in an amount from 1×10⁷ CFU to 1×10¹¹ CFU and
a second composition comprising resveratrol preferably in an amount from 10 to 300 mg and a curcuma extract in an amount from 1 to 300 mg, e.g. from 10 to 300 mg, said kit preferably being suitable for use in the management of subjects who suffer from or are likely to suffer from endometriosis.

The combination, composition or kit according to the invention may be characterized in that:
the Saccharomyces cerevisiae yeast is a Saccharomyces cerevisiae of the variety boulardii, and/or
the resveratrol extract is added in the form of a plant extract enriched with trans-resveratrol, for example an extract of Japanese knotweed titrated to at least 95% by weight of trans-resveratrol and/or
the curcuma extract comprises at least 15% by weight of curcuminoids, and/or
the curcuma extract comprises curcumin, demethoxycurcumin and bisdemethoxycurcumin, and/or
the curcuma extract comprises at least 55% by weight, preferably at least 60% by weight of curcumin relative to the total weight of the curcuminoids present in the extract, and/or
the curcuma extract is encapsulated in a cyclodextrin, preferably a gamma cyclodextrin, and/or
the curcuma extract is combined with an encapsulation or vectoring system, preferably a cyclodextrin.

The invention also relates to the use of resveratrol, a curcuma extract and a Saccharomyces cerevisiae yeast in combination for preparing a nutraceutical or pharmaceutical composition for treating a subject suffering from endometriosis or susceptible to endometriosis, and more precisely for treating or preventing endometriosis or a symptom thereof.

The invention further relates to a method for treating a subject suffering from endometriosis or is susceptible to endometriosis comprising the administration of an effective dose of resveratrol, a curcuma extract and Saccharomyces cerevisiae yeast to said subject.

### DESCRIPTION OF THE FIGURES

FIG. 1 shows the growth, the weight and the volumes of murine endometriotic lesions over time. M (control): group treated with a mixture of resveratrol and curcuma extract; M1 (invention): group treated with a mixture of resveratrol, curcuma extract and S. boulardii; M2 (invention): group treated with a mixture of resveratrol, curcuma extract, S. boulardii and L. acidophilus.

FIG. 2 shows the histology of lesions sampled at sacrifice (HE staining) for the different groups M, M1 and M2.

FIG. 3 shows the splenic immune state of the different groups after 12 weeks of treatment (S12)— T lymphocytes. SHAM (control) corresponds to the animals that received sham surgery; M (control): group treated with a mixture of resveratrol and curcuma extract; M1 (invention): group treated with a mixture of resveratrol, curcuma extract and S. boulardii; M2 (invention): group treated with a mixture of resveratrol, curcuma extract, S. boulardii and L. acidophilus.

FIG. 4 shows the splenic immune state of the different groups after 12 weeks of treatment (S12)— B Lymphocytes and Macrophages. SHAM corresponds to the animals that received sham surgery;

M (control): group treated with a mixture of resveratrol and curcuma extract; M1 (invention): group treated with a mixture of resveratrol, curcuma extract and S. boulardii; M2 (invention): group treated with a mixture of resveratrol, curcuma extract, S. boulardii and L. acidophilus.

FIG. 5 shows the effect on zonulin, interleukin 6 (IL6), TNFα and the advanced oxidation protein products (AOPP). SHAM (control) corresponds to the animals that received sham surgery; M (control): group treated with a mixture of resveratrol and curcuma extract; M1 (invention): group treated with a mixture of resveratrol, curcuma extract and S. boulardii; M2 (invention): group treated with a mixture of resveratrol, curcuma extract, S. boulardii and L. acidophilus.

### DETAILED DESCRIPTION OF THE INVENTION

Endometriosis is a disease characterized by the presence of ectopic endometrium that forms cystic lesions on the ovaries and in the peritoneal cavity. Little is known about the physiopathology of this disease. Recent studies suggest that endometriosis is an immunologic, estrogen-dependent inflammatory or even autoimmune disease (Ahn, 2016; Khan, 2010; Lin, 2016; Malutan, 2015). It has also been mentioned that dysbiosis and/or an intestinal and vaginal hyperpermeability as well as oxidative stress would have an impact on the progression of the disease (Baker, 2017; Laschke, 2015; Puca, 2017; Ito, 2017; Scutiero, 2017).

The inventors have shown, surprisingly, that the combined administration of a yeast Saccharomyces cerevisiae, resveratrol and a curcuma extract (Curcumalonga) enabled to significantly slow-down the progression of the endometriotic lesions in an animal model of endometriosis, relative to the control animals and to animals treated with a combination of resveratrol and a curcuma extract, after 12 weeks of treatment.

The inventors have also shown that this combination of active ingredients made it possible to modulate the immune state by increasing the proportion of type 1 macrophages relative to type 2 macrophages. Such an effect was not observed in the control animals or those treated with a combination of resveratrol and a curcuma extract.

Thus, the inventors have shown that the three active ingredients according to the invention, namely the yeast Saccharomyces cerevisiae, resveratrol and curcuma extract, exert a synergistic action making it possible to stop the endometriotic lesions or even cause them to regress, and to modulate a patient's immune state.

The inventors have also shown that the combined administration of resveratrol, curcuma extract and a yeast Saccharomyces cerevisiae made it possible to reduce the production of pro-inflammatory cytokines such as TNFα and interleukin 6, as well as cellular oxidative stress. Notably, this combination of active ingredients also made it possible to lower the plasma level of zonulin, a protein involved in intestinal hyperpermeability, a biomarker of low-grade inflammatory reactions.

To summarize, combined administration of resveratrol, a curcuma extract and a yeast Saccharomyces cerevisiae makes it possible to prevent or treat the development of endometriotic lesions. This combination of active ingredients also makes it possible to modulate the immune system, in particular to adjust the macrophages ratio M1/M2, and normalize the inflammatory state at the peritoneal level in a subject with endometriosis, in particular by reducing oxidative stress and intestinal permeability.

Thus, the invention relates to the use of resveratrol, a curcuma extract and Saccharomyces cerevisiae yeast in combination for treating a subject suffering from endometriosis or susceptible to endometriosis. More precisely, the invention relates to the use of resveratrol, a curcuma extract and Saccharomyces cerevisiae yeast in combination in the treatment or prevention of endometriosis or a symptom thereof or associated disorders.

The invention further relates to the use of resveratrol, a curcuma extract and Saccharomyces cerevisiae yeast in combination for preparing a composition, preferably nutraceutical, food or pharmaceutical composition, for treating a subject suffering from endometriosis or susceptible to endometriosis, and more precisely for treating or preventing endometriosis or a symptom thereof.

The invention also relates to a method of treatment of a subject suffering from endometriosis or is susceptible to endometriosis comprising the administration of an effective dose of resveratrol, a curcuma extract and Saccharomyces cerevisiae yeast to said subject. The invention also relates to a kit or a composition, preferably pharmaceutical, nutraceutical or food composition, comprising the combination of resveratrol, a curcuma extract and Saccharomyces cerevisiae yeast, said composition or said kit being suitable for subjects suffering from endometriosis or subjects susceptible to endometriosis.

### General Definitions

As used here, the terms “disorder”, “anomaly” or “affection” are interchangeable and refer to an organ, a part, a structure or a system of the body that is functioning incorrectly. Preferably, the term “affection” denotes a health affection, for example disturbing the normal physical or mental functions.

As used here, a “pharmaceutical composition” denotes a preparation of one or more active agents with other optional chemical components such as physiologically suitable carriers and/or excipients. The purpose of a pharmaceutical composition is to facilitate the administration of the active agent to an organism. The compositions of the present invention may be in a form suitable for any route of administration or conventional use. The pharmaceutical composition generally comprises a pharmaceutically acceptable vehicle.

As used here, the terms “pharmaceutically acceptable carrier” or “pharmaceutically acceptable excipient” or “pharmaceutically acceptable vehicle” are interchangeable and comprise all compounds or combinations of compounds that are known by a person skilled in the art to be useful in the formulation of pharmaceutical or veterinary compositions. In the context of the present invention, “physiologically acceptable” or “pharmaceutically acceptable” means any medium or additive that does not interfere with the efficacy of the biological activity of the active pharmaceutical ingredient (here, the combination of resveratrol, curcuma extract and yeast S. cerevisiae), and that is not excessively toxic for the subject, at the concentrations at which it is administered and/or that does not produce an undesirable reaction when it is administered. A physiologically acceptable vehicle, carrier or excipient may be suitable for administration to humans and/or to animals (in particular to mammals).

The term “medicinal product”, as used here, includes medicinal products for human use in human medicine and refers to any pharmacologically acceptable substance that provides a therapeutic and/or beneficial effect. The term “medicinal product” used here is not necessarily limited to substances requiring marketing authorization, but may comprise substances that may be used in cosmetics and nutraceutical compositions, for example food supplements.

The term “nutraceutical” refers to a composition or product manufactured from food substances, but made available in the form of tablets, powder, potion or other galenical forms not usually associated with foodstuffs, and having a beneficial physiological effect or protective effect against human affections or diseases. In particular, this definition includes food supplements, certain foodstuffs for a specific group or meal replacements.

“Nutraceutical composition” means any composition comprising food ingredients such as macronutrients, micronutrients, plants or plant extracts, or substances having a nutritional or physiological effect, with the aim of supplementing the diet of a human being in order to improve the latter's nutritional state and thus promote a good state of health. As envisaged here, the nutraceutical products are isolated nutrients, food supplements or are included in certain foodstuffs intended for specific groups. The nutraceutical compositions thus include for example the combination according to the invention and, optionally, a food-grade component, in particular a processing additive or aid.

Here, “food supplement” means any composition that is formulated and administered separately from other foodstuffs and has the aim of supplementing a subject's nutritional intake having a pharmacologically acceptable form, in particular in the form of hard capsules, tablets, soft capsules, sachets, stick packs, sirup, oral solution packaged in ampules, a dropper, or any other suitable form familiar to a person skilled in the art.

The terms “good”, “food product” and “foodstuff” are used interchangeably here and comprise, in addition to the foodstuffs commonly consumed by humans, functional foods and foods for specific groups, themselves comprising foodstuffs intended for special medical purposes.

“Food composition” means any composition comprising food ingredients such as macronutrients, micronutrients, vitamins, minerals, trace elements or substances having a nutritional or physiological effect. As envisaged here, the food products are conventional foodstuffs, functional foods or transformed foods. Thus, by definition, the food compositions may be various foodstuffs known by a person skilled in the art.

The food composition may in particular be a food intended for human nutrition, which may be a liquid, a paste or a solid. As examples, without being limited thereto, the food may be a milk product such as cheese, butter, cream, yoghurts, ice creams, cheeses, baked products such as bread, biscuits and cakes, a product based on fruit such as a fruit juice, a fruit salad or a fruit paste, food products based on soybean, food products based on starch, confectionery products, compositions for edible oils, spreadable pastes, breakfast cereals, food bars; biscuits, snacks, chewing gums; drinks; energy drinks, enriched drinks; drink supplements (powders to be added to a drink); etc.

According to a particular aspect, the food composition is not based on milk. Preferably, the food composition does not comprise protein(s) or oligosaccharide(s) of human or animal milk. For example, the food composition according to the invention comprises the combination of active substances according to the invention and the components of at least one of the foodstuffs or drinks mentioned above.

Here, the term “functional food” refers to a conventional foodstuff that forms part of the normal diet and provides physiological benefits and/or reduces the risk of diseases or disorders and/or reduces the symptoms associated therewith, besides the conventional nutrients that it contains. As used here, the term “food additive” refers to a compound or a composition that is intended to be mixed with one or more other foodstuffs before being administered to a subject. More particularly, “food additive” means any additive as defined by the Codex alimentarius, General standard for food additives—Codex Stan 192-1995, i.e. any substance that is not consumed alone as a foodstuff, nor is used alone as an ingredient typical of a foodstuff, whether or not it is of nutritional value, and whose intentional addition to a foodstuff for a technological (including organoleptic) purpose at any step in the manufacture, processing, preparation, treatment, packaging, wrapping, transport or storage of said foodstuff involves, or may in all probability involve, directly or indirectly, the incorporation thereof or of its derivatives in this foodstuff or may affect its characteristics in some other way.

As used here, a “prebiotic” refers to an ingredient or substrate which, used selectively by a microorganism of the host, confers a health benefit. It may allow beneficial changes to be induced, both in the composition and/or the activity of a probiotic. A prebiotic may be an edible food or drink or an ingredient thereof. The prebiotic may include complex carbohydrates, polyphenols and polyunsaturated fatty acids. A prebiotic is usually an indigestible carbohydrate such as an oligo- or polysaccharide, or a sugar alcohol, which is not degraded or absorbed in the upper alimentary tract in the absence of intestinal microorganisms. Preferably, the prebiotics according to the invention are as defined in the Decree of 26 Sep. 2016 establishing the list of substances with a nutritional or physiological purpose permitted in food supplements and the conditions for use thereof.

As used here, the term “probiotic” refers to live bacteria or to inactivated bacteria which, when administered in suitable amounts, have a beneficial effect on the host organism. These compositions are advantageous in that they are suitable for administration to humans and to other mammalian subjects. The probiotic substances contain a sufficiently high number of probiotic microorganisms to exert a direct or indirect action on the intestinal microbiota. It should be noted that, for the purposes of the present description, “probiotic” means any biologically active form of probiotic, preferably, but not exhaustively, yeasts, lactobacilli, bifidobacteria, bacilli, streptococci, enterococci, propionibacteria or saccharomycetes but also other microorganisms making up the “normal intestinal flora”, or else fragments of the bacterial wall or of the DNA of these microorganisms. Preferably, the probiotics according to the invention are as defined by the Food and Agriculture Organization of the United Nations (FAO) and the World Health Organization (WHO) in the Mixed Consultation of FAO/WHO experts for evaluating the health and nutritional properties of probiotics in foodstuffs, including milk powder containing live lactic acid bacteria conducted in 2001.

The terms “microbiota” or “microflora” used hereinafter are interchangeable and denote all of the microbial species present (permanently or temporarily) in an environment, comprising eukaryotes, archaea, prokaryotes and viruses. The term “microbiome” refers to the “genetic contents” of the microbiota and comprises genomic DNA, ribosomal RNA, the epigenome, plasmids and all other types of genetic information of the microbiota. Preferably, these terms refer here to bacteria of the human intestinal microbiota.

As used here, the term “yeast” denotes microscopic fungi, with ovoid cells with a width of a few thousandths of millimeters, easily detachable from one another and accordingly well adapted to propagation in liquids. The term “yeast” includes all the species of the genus Saccharomyces.

As used here, the term “bacterium” denotes any prokaryotic microorganism existing in the form of a single cell, in a group or in an aggregate of individual cells. The term “bacterium” encompasses all the variants of bacteria (for example, endogenous bacteria or environmental bacteria), more particularly the bacteria of the human or animal microbiota, in particular the intestinal microbiota.

As used here, the term “comprising” or “comprises” is used in reference to substances, compounds or processes that are essential to the invention, but that are open to the inclusion of nonspecific elements, whether essential or not. The use of “comprising” indicates inclusion rather than limitation.

The term “and/or” as used here is to be regarded as a specific description of each of the two characteristics or components specified, with or without the other one. For example, “A and/or B” is to be regarded as a specific disclosure of each of the following elements: (i) A, (ii) B and (iii) A and B, as if each of them was presented individually.

The term “about” as used here in relation to all the values (including the lower and upper limits of numerical ranges) signifies any value having an acceptable variation of up to ±10% (for example, ±0.5%, ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%). The use of the term “about” at the beginning of a list of values modifies each of them (i.e. “about 1, 2 and 3” refers to about 1, about 2 and about 3). Furthermore, when a list of values is described (for example about 50%, 60%, 70%, 80%, 85% or 86%), the list includes all its intermediate and fractional values (for example 54% or 85.4%).

The term “consists essentially of” as used here to define a composition in which the elements other than those mentioned do not represent more than 5% by weight of the composition, preferably not more than 3% by weight, and more specifically not more than 1% by weight.

Uses According to the Invention

According to a first aspect, the invention relates to the use of resveratrol, a curcuma extract and Saccharomyces cerevisiae yeast in combination in the management of a subject suffering from endometriosis or susceptible to suffer from endometriosis.

The combination according to the invention may be used for treating a subject with endometriosis. More precisely, the invention relates to the use of resveratrol, a curcuma extract and yeast Saccharomyces cerevisiae in combination in the treatment or prevention of endometriosis or a symptom thereof or associated disorders.

The invention relates in particular to the use of a combination of resveratrol, a curcuma extract and yeast Saccharomyces cerevisiae for treating or preventing endometriotic lesions. More particularly, the combination according to the invention may be used for preventing or slowing the development of endometriotic lesions, or for decreasing the volume or weight of endometriotic lesions in a subject suffering from endometriosis or in a subject susceptible to endometriosis.

According to a certain aspect, the combination of resveratrol, a curcuma extract and yeast Saccharomyces cerevisiae according to the invention is used for preventing or treating, e.g. reducing, chronic peritoneal inflammation in a subject suffering from endometriosis or susceptible to endometriosis.

According to an additional aspect, the combination of resveratrol, a curcuma extract and yeast Saccharomyces cerevisiae according to the invention is used for preventing or treating, e.g. reducing intestinal hyperpermeability, or for normalizing the intestinal permeability in a subject suffering from endometriosis or susceptible to endometriosis.

According to another aspect, the combination of resveratrol, a curcuma extract and yeast Saccharomyces cerevisiae according to the invention is used for normalizing or modulating the immune state, e.g. promoting normal functioning of the immune system, in a subject suffering from endometriosis or susceptible to endometriosis.

According to an additional aspect, the combination of resveratrol, a curcuma extract and yeast Saccharomyces cerevisiae according to the invention is used for preventing or decreasing oxidative stress at the peritoneal level in a subject suffering from endometriosis or susceptible to endometriosis.

According to another aspect, the combination of resveratrol, a curcuma extract and yeast Saccharomyces cerevisiae according to the invention is used for preventing or treating one or more symptoms or disorders associated with endometriosis.

The combination according to the invention may be used, for example, for preventing or reducing the frequency and/or intensity of pains such as pelvic pains such as dysmenorrhea and dyspareunia and catamenial pains for example at the intestinal or urinary level in a subject suffering from endometriosis or is susceptible to endometriosis.

The combination according to the invention may also be used for treating or preventing hypofertility or infertility, in a subject who has endometriosis or is likely to suffer from endometriosis. The combination according to the invention may also be used for improving fertility, or maintaining a normal fertility, in a subject suffering from endometriosis or susceptible to endometriosis.

The combination according to the invention may be used for maintaining fertility in a subject suffering from endometriosis or in a subject susceptible to endometriosis. The combination according to the invention may in particular be used for normalizing or improving the hormonal state of a subject suffering from endometriosis or in a subject susceptible to endometriosis, for example normalizing or improving the plasma level of AMH (antimüllerian hormone) and/or estradiol.

According to an additional aspect, the combination according to the invention may be used for improving the general condition of a subject suffering from endometriosis or susceptible to endometriosis.

“Improving the general condition” means improving the physiological and/or psychological state of a subject, which may lead in particular to an improvement in their quality of life. The combination according to the invention may make the disease and the associated symptoms more bearable. It may be a nontherapeutic use of the combination according to the invention.

Thus, according to an additional aspect, the invention relates to the nontherapeutic use of a combination of resveratrol, a curcuma extract and yeast Saccharomyces cerevisiae as an agent for improving the wellbeing and/or quality of life of a subject having or susceptible to suffer from endometriosis. In particular, the invention relates to the nontherapeutic use of a combination of resveratrol, a curcuma extract and Saccharomyces cerevisiae yeast as a food supplement in the management of a subject having or susceptible to suffer from endometriosis.

1. Endometriosis and Associated Symptoms and Disorders, Subject According to the Invention

As stated in the technological background section, endometriosis is a pathology that is characterized by the presence of endometrial tissue outside the uterine cavity. This tissue may be present in particular at the level of the walls of the uterus, fallopian tubes, ovaries, uterosacral ligaments but also at the level of the bladder and colon.

In the sense of the invention, the term “endometriosis” encompasses all the known forms of endometriosis, including adenomyosis, endometriomas, deep endometriosis, or the endometrioses of stages Ito IV according to the classification of the American Fertility Society. encompass, without being limited to, pains, abnormal or excessive bleeding such as proctorrhagia, blood in the urine and metrorrhagia, digestive or urinary disorders such as diarrhea, constipation, frequent and/or painful urination in particular at the time of menstruation, chronic fatigue, hypofertility and infertility. The symptoms and disorders associated with endometriosis also encompass chronic peritoneal inflammation, intestinal hyperpermeability, and vaginal and/or intestinal dysbiosis.

The pains associated with endometriosis are varied and include in particular dysmenorrhea (pelvic pains during menstruation), lumbar pains, dyspareunia and catamenial pains in particular at the urinary or intestinal level.

In the context of the present invention, “hypofertility” means that a woman has not become pregnant after at least 6 months of regular sexual relations without contraception. “Infertility” means that a woman has not become pregnant after 24 months of regular sexual relations without contraception.

“Regular sexual relations” corresponds to sexual intercourse about at least once every 48 h. In a particular embodiment, the combination of active substances according to the invention is used for treating or preventing a symptom or disorder associated with endometriosis selected from the group consisting of dysmenorrhea, dyspareunia, chronic fatigue, hypofertility, infertility and combinations thereof.

In the context of the invention, the term “treat, treatment” means obtaining a desired pharmacological and/or physiological effect. The effect may be prophylactic in terms of complete or partial prevention of a pathology or an associated disorder or symptom thereof and/or may be therapeutic in terms of partial or complete cure of said disease. The term “treatment” as used here covers any treatment of a disease in a subject of interest, for: reducing the incidence and/or risk of recurrence of the disease or disorder for a length of time without symptoms; alleviating or reducing a symptom or a disorder associated with the disease; preventing the disease or an associated symptom or disorder or the disorder developing in a subject who suffers from or is predisposed to the disease; stopping, slowing or delaying the development of the disease or an associated symptom or disorder; reducing the frequency and/or the intensity of episodes of the disease, for example the symptoms; and alleviating the disease or an associated symptom or disorder, i.e. causing complete or partial regression of said disease, associated symptom or disorder.

As an example, the combination according to the invention may be used for preventing (e.g. preventing, slowing, or delaying) the appearance and the development of endometriotic lesions and/or reducing, or even treating, the endometriotic lesions.

As an additional example, the combination according to the invention may be used for treating, e.g. decreasing the frequency and/or intensity of pelvic pains in a woman suffering from endometriosis.

The subject according to the invention is a menstruating woman, of any age in particular between 12 years and 50 years, preferably between 14 years and 45 years.

A “subject suffering from endometriosis” means a woman for whom the presence of at least one endometriotic lesion has been diagnosed, for example during clinical examination, by an imaging technique (ultrasonography, MRI) or by celioscopy. A “woman suffering from endometriosis” includes in particular a woman in remission, for example a woman having undergone medical or surgical treatment with the aim of eliminating or decreasing the endometriotic lesions.

The subject suffering from endometriosis may be receiving treatment for the management of endometriosis or a symptom thereof, including the taking of contraceptives, in particular oral contraceptives, NSAIDs, and treatments used in medically assisted reproduction, in particular ovarian stimulation treatments, or even hydrotherapy. It may also be a treatment by alternative medicine (osteopathy, acupuncture, kinesitherapy, self-hypnosis, sophrology . . . )

A woman suffering from endometriosis may have one or more of the disorders or symptoms described above. In a preferred embodiment, a woman suffering from endometriosis has at least one symptom or disorder selected from dysmenorrhea, dyspareunia, chronic fatigue, infertility and combinations thereof.

“A subject susceptible to endometriosis” or “a subject predisposed to endometriosis” means a woman having a clinical picture suggesting the presence of endometriosis, for example showing strong or severe pains during menstruation, and/or hypofertility or infertility and/or having a family history of the disease (for example a mother, an aunt, a grandmother and/or a sister suffering from endometriosis).

2. Combination of Active Ingredients According to the Invention

The combination of active ingredients according to the invention comprises resveratrol, a curcuma extract and a Saccharomyces cerevisiae yeast.

According to the invention, “combination of active ingredients” or else “association of active ingredients” means a set of active substances that may act interactively or synergistically to obtain one or more pharmacological, and/or physiological and/or nutritional effects with the aim of improving the general condition of a subject and/or treating or preventing a pathology or an associated symptom or disorder thereof. As explained above, the combination of active ingredients according to the invention finds application in the management of subjects suffering from endometriosis or subjects susceptible to endometriosis.

Resveratrol

Resveratrol is a polyphenol of the stilbene class present in many plant species. In the sense of the invention, the term resveratrol encompass both the trans isomer and the cis isomer. In certain embodiments, resveratrol is in the form of a mixture of trans and cis isomers. In other embodiments, resveratrol is in the form of the trans isomer. It is therefore trans-resveratrol of CAS No. 501-36-0.

Preferably, resveratrol is present in the form of trans-resveratrol.

Resveratrol may be prepared by chemical synthesis or may be purified starting from any plant or plant part known to comprise resveratrol such as the berries, pips, leaves and shoots, of grapevine, Japanese knotweed, cocoa, cranberry, mulberries, or also nuts.

Resveratrol may be supplied in the pure form, i.e. having a purity of at least 99%.

If applicable, resveratrol may be supplied in the form of a resveratrol-enriched plant extract, i.e. having at least 50% (e.g. at least 60%, 70%, 80%, 85%, 90% or 95%) by weight of resveratrol relative to the weight of dry matter of the extract.

In a particular embodiment, resveratrol is present in the form of an extract of Japanese knotweed (Fallopia japonica (Houtt.) Ronse Dec., polygonaceae) titrated to at least 95% by weight, e.g. at least 98% by weight of trans-resveratrol relative to the total weight of the composition.

According to the invention, the daily dose of resveratrol may be in the range from 10 mg to 300 mg, for example from 25 mg to 250 mg, from 25 mg to 100 mg, preferably from 30 mg to 70 mg or from 40 mg to 60 mg.

The Yeast Saccharomyces cerevisiae

In the sense of the invention, “yeast Saccharomyces cerevisiae” or “Saccharomyces cerevisiae yeast” means any yeast belonging to the species Saccharomyces cerevisiae. The species Saccharomyces cerevisiae includes in particular the variety boulardii and the variety cerevisiae. Preferably, the invention is performed with a yeast Saccharomyces cerevisiae belonging to boulardii Variety.

In a particular embodiment, the yeast Saccharomyces cerevisiae is selected from the group consisting of the strains of Saccharomyces cerevisiae var. boulardii, the strains of Saccharomyces cerevisiae var. cerevisiae and combinations thereof.

In a preferred embodiment, the yeast Saccharomyces cerevisiae is selected from the strains belonging to the variety Saccharomyces cerevisiae boulardii.

Several strains of Saccharomyces cerevisiae, in particular of the boulardii variety, are available commercially.

We may mention in particular the strain marketed by Lesaffre Human Care under the trademark Lynside® Pro SCB and registered under CNCM (National Collection of Cultures of Microorganisms) number 1-3799.

As an additional example, we may also mention the strain with CNCM number 1-745 deposited on 28 Mar. 1988 with the CNCM-Pasteur Institute by the Biocodex Laboratories and available commercially, for example in the Ultra-levure® products. We may also mention the strain with CNCM number 1-1079 marketed by the company Lallemand or the strains deposited under ATCC (American Type Culture Collection) No. MYA-796 or MYA-797.

It is to be understood that the present invention also relates to the use of any yeast of the species Saccharomyces cerevisiae, preferably of the boulardii variety as well as the use of any strain or of homologs obtained from this bacterial species. The terms “homolog”, “variant” or “mutant” are interchangeable and refer to a bacterial strain having homology or sequence identity with the nucleotide sequence of the parent bacterial strain (reference sequence). This includes strains having at least 95%, 96%, 97%, 98% or 99% identity with the nucleotide sequence of the genome of the species Saccharomyces cerevisiae, preferably with the nucleotide sequence of the genome of the strain Saccharomyces cerevisiae var. boulardii registered under CNCM number 1-745 or that registered under CNCM number 1-3799. The mutants may be obtained by genetic engineering techniques tenabling the alteration of the genetic material of the strains of the invention or to infer a recombination of the genetic material of the strains of the invention with other molecules. Typically, in order to obtain mutant strains of this kind, a person skilled in the art may use standard mutagenesis techniques such as UV radiation or exposure to mutagenic chemicals.

The yeast Saccharomyces cerevisiae present in the combination according to the invention may be alive or inactivated. Preferably, the yeast is in the living form. The yeast Saccharomyces cerevisiae may in particular be in a hydrated, lyophilized, frozen, sprayed form or any other form allowing it to be administered alive to the subject. The yeast may be administered as it is or after being taken up in a suitable physiologically acceptable vehicle.

The yeasts are administered, for example, in the form of cultured live bacteria, in the vegetative form or in the form of spores. Alternatively, the yeasts are supplied in the form of purified populations obtained from a microbiotic material such as faecal material. “Live/alive” is to be understood as meaning that the integrity of the cell is preserved and that the cellular processes take place or may take place if the yeast is cultured in a suitable medium and suitable conditions. Any live yeast may be reseeded on a suitable culture medium and is able to multiply in suitable conditions.

In certain embodiments, the yeast Saccharomyces cerevisiae is administered at a dose from 10⁷ to 10¹¹, in particular from 10⁷ to 10¹⁰, preferably from 10⁸ to 10¹⁰ or from 10⁸ to 10⁹ colony-forming units (CFU).

As an example, the yeast Saccharomyces cerevisiae is administered at a dose by weight from 5 to 100 mg.

Preferably, it is a strain belonging to the variety Saccharomyces cerevisiae var. boulardii, for example that deposited under CNCM No. 1-3799 or 1-745.

Preferably, the combination according to the invention comprises the daily administration of from 2×10⁸ to 6×10⁸ CFU, more preferably from 3×10⁸ to 5×10⁸ CFU of Saccharomyces cerevisiae var. boulardii.

The Curcuma Extract

According to the invention, Curcuma longa L., also called curcumin, curcuma or turmeric, is a perennial plant originating from Asia, which belongs to the Zingiberaceae family. The rhizome of curcuma consists of mucilage, polysaccharides, essential oils, polyphenols, and curcuminoids, in particular giving the rhizome its yellow/orange color.

“Curcuminoids” are compounds of the diarylheptanoid type comprising in particular curcumin and derivatives thereof such as demethoxycurcumin or bis-demethoxycurcumin.

Curcumin (1,7-bi s(4-hydroxy-3-methoxyphenyl)-1,6-heptadiene-3,5-dione), also called diferuloylmethane (CAS No.: 458-37-7), is the principal curcuminoid of Asian curcuma.

The curcuma extract present in the combination is typically a rhizome extract, dry or fresh, Curcuma longa L. It may be an extract standardized for curcuminoids.

In the sense of the invention, the term “standardized” or “standardize” means the concept of controlling an extract, an oil or any other ingredient so that it complies with a standard or with a defined minimum content of certain molecules, for example such as curcuminoids in the case of curcuma extract. A curcuma extract standardized for curcuminoids is therefore a curcuma extract having a defined minimum content of curcuminoids, preferably a minimum content of 15% of curcuminoids.

Thus, the curcuma extract according to the invention comprises a content of at least 15% by weight of curcuminoids relative to the total weight of the extract, preferably relative to the total weight of dry matter of the extract.

The curcuma extract present in the combination according to the invention may be obtained by any known method of extraction, for example by extraction using an organic solvent, by maceration or decoction or else by extraction with supercritical fluid. Preferably it is a curcuma rhizome extract obtained by a method comprising a step of extraction with a solvent usable in the preparation of food supplements, for example an alcohol or an ester such as ethyl acetate. Advantageously, the curcuma extract according to the invention is in the form of powder. Its water content is preferably below 15% (e.g. below 10% or 5%) relative to the total weight of the extract. According to a preferred embodiment of the invention, the curcuma extract contains at least 10, 15, 20, 25, 30, 35, 40 or 45% by weight of curcuminoids.

In a particular embodiment, the curcuma extract comprises curcumin, demethoxycurcumin and/or bisdemethoxycurcumin. Preferably, curcumin is the predominant curcuminoid in the extract. Curcumin may represent at least 55% by weight, preferably at least 60% by weight relative to the total weight of the curcuminoids present in the extract.

As an example, the curcuma extract comprises curcumin, demethoxycurcumin and bisdemethoxycurcumin in the following proportions: from 65 to 82% of curcumin, from 15 to 25% of demethoxycurcumin and from 2 to 7% of bisdemethoxycurcumin, the percentages being expressed by weight relative to the total weight of curcuminoids present in the extract.

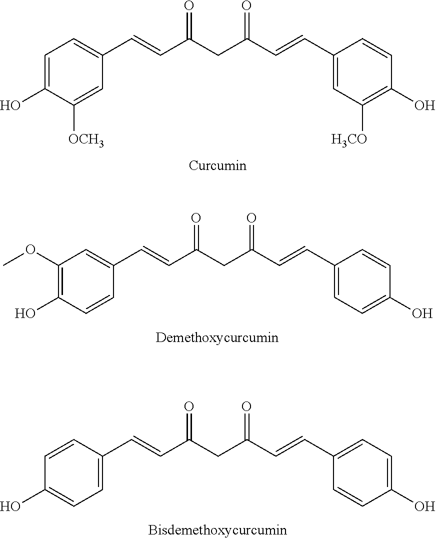 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(O)cc2)ccc1O ; O=C(/C=C/c1ccc(O)cc1)CC(=O)/C=C/c1ccc(O)cc1]

In order to improve the bioavailability of the curcuminoids, the curcuma extract may be formulated using a carrier or an encapsulation system such as liposomes or cage molecules. In a particular embodiment, the curcuma extract is combined with a cyclodextrin, preferably a gamma-cyclodextrin. It may in particular be the product CAVACURMIN® marketed by Wacker.

According to a particular aspect, the combination according to the invention comprises curcumin introduced into the composition by adding a curcuma extract or in a purified form. According to an additional aspect, the combination according to the invention comprises the daily administration of from 1 to 100 mg of curcuminoids, preferably from 5 to 50 mg of curcuminoids, for example from 10 to 30 mg or from 10 to 20 mg of curcuminoids, said curcuminoids comprising at least curcumin, and preferably curcumin, demethoxycurcumin and bisdemethoxycurcumin, and said curcuminoids being administered in the form of a curcuma extract.

Preferably, the curcuma extract is as described above. Preferably, the curcuma extract is combined with a gamma-cyclodextrin and the weight ratio of curcuminoids to gamma-cyclodextrin is within a range from 1 to 10, preferably from 2 to 8 and more preferably from 3 to 7, for example from 4 to 5.

3. Regimen for Administration of the Combination According to the Invention

The combination or association according to the invention may be administered to the subject of interest by any suitable route, for example by the buccal, oral, nasal, transmucosal for example vaginal, intrauterine, intrarectal, or transdermal route.

Preferably, the combination according to the invention is administered by the oral route.

The ingredients of the combination according to the invention, namely resveratrol, curcuma extract and the yeast Saccharomyces cerevisiae may be administered to the subject simultaneously. For example, resveratrol, curcuma extract and the yeast Saccharomyces cerevisiae may be present in the same pharmaceutical, nutraceutical or nutritional composition. Alternatively, resveratrol, curcuma extract and the yeast Saccharomyces cerevisiae may be administered separately, successively or separately over time. For example, resveratrol, curcuma extract and the yeast Saccharomyces cerevisiae may be administered in the form of a kit, i.e. in the form of several separate pharmaceutical, nutraceutical or nutritional compositions, for example in the form of at least two dosage units for oral administration, for example a first capsule containing the yeast and a tablet or a second capsule containing resveratrol and curcuma extract. The kit for administering the combination may alternatively comprise three separate capsules, a capsule containing yeast, a tablet or capsule containing resveratrol, and a tablet or capsule containing curcuma extract.

The doses, frequency of administration and the duration of the treatment depend on the subject's characteristics, in particular her age, weight, medical history, the stage and the severity of endometriosis or of the associated symptoms or disorders, the medical treatments received, etc . . . .

Typically, the combination according to the invention may be administered at least once per month, per week or per day. Preferably, the combination according to the invention is administered daily for at least one week, typically for at least two weeks, at least three weeks, at least four weeks, at least for 30 days, or even for at least 2, 3, 4, 6, 12 months or even longer. As an example, the combination according to the invention may be administered daily for 1 to 3 months, and this treatment may be repeated if needed.

The combination according to the invention is typically administered in an effective or sufficient dose to obtain one of the expected effects, as described above, in particular an effect enabling to decrease, slow or treat a disorder or symptom associated with endometriosis in particular in connection with the development of endometriotic lesions and/or enabling to improve the general condition of a subject with endometriosis.

As an example, in a method of treatment of endometriosis, a “therapeutically effective amount” of a composition may correspond to an amount that reduces the severity of a symptom and/or reduces a measurable parameter associated with the pathology by at least about 5%, 10%, at least about 20%, at least about 30%, at least about 40%, at least about 50%, at least about 60%, or more, in comparison with the symptom (for example, the severity of the symptom), or in comparison with the measurable parameter, in the absence of treatment of the subject. For example it may be an effect of slowing or of decreasing the endometriotic lesions that can be visualized for example by imaging (ultrasonography, MM and other tomography techniques), an effect of improvement in fertility or a decrease in measurable inflammation, for example by means of certain blood markers or else a decrease in the intensity of the pains measurable using an analog pain scoring scale.

Typically, the daily doses to be administered may range:
from 10 to 300 mg, preferably from 25 to 250 mg of resveratrol,
from 1 to 300 mg, for example from 10 to 300 mg, from 25 to 250 mg of a curcuma extract, or preferably from 5 to 25 mg of curcuma extract and
from 1×10⁷ CFU to 1×10¹¹ CFU, preferably from 1×10⁸ CFU to 1×10¹⁰ CFU or from 1×10⁸ CFU to 1×10⁹ CFU or from 5 to 100 mg, or even from 5 to 50 mg of yeast Saccharomyces cerevisiae, preferably of the boulardii variety.

As an additional example, the daily doses to be administered may range:
from 30 mg to 70 mg, preferably from 40 mg to 60 mg of resveratrol,
from 5 to 100 mg, preferably from 5 to 25 mg of curcuminoids in the form of curcuma extract, and
from 2×10⁸ CFU to 6×10⁸ CFU, preferably from 3×10⁸ CFU to 5×10⁸ CFU of yeast Saccharomyces cerevisiae, preferably of the boulardii variety.

These daily doses may be administered in one, two, three or four separate doses per day. Preferably, the combination according to the invention is administered in one or two separate doses per day, preferably once per day.

The composition according to the invention may be administered by oral route before a meal. In an additional embodiment, the combination according to the invention is administered in combination with one or more conventional medicinal products used for the management of endometriosis and the associated symptoms. These medicinal products encompass, without being limited to, contraceptives, in particular oral contraceptives of the progestogen pill or combined pill type, analgesics, in particular acetaminophen and aspirin, NSAIDs, in particular ibuprofen and flurbiprofen, or else ovarian stimulation treatments used in medically assisted reproduction (MAR), in particular in the context of an in-vitro fertilization protocol.

The combination according to the invention may also be used in the management of endometriosis in combination with surgery for resection of endometriotic lesions. As an additional example, the combination according to the invention may also be used in the management of endometriosis in combination with a hydrotherapy. Here, “hydrotherapy” means the use of natural mineral waters from hot springs and products derived from them thereof (for example thermal gases and muds). As an example, the hydrotherapy may be carried out for at least three consecutive weeks, for example with the aim of alleviating the pains associated with endometriosis, and comprises in particular the application of treatments such as vaginal irrigation, mother liquor compresses, showers with thermal water at variable pressure, and mobilization sessions in a thermal water swimming pool.

More generally, the combination according to the invention may be used in combination with an alternative medicine treatment, for example in combination with acupuncture, osteopathy, yoga, kinesitherapy, sophrology, naturopathy, self-hypnosis, meditation and/or relaxation. In a particular embodiment, the combination according to the invention is administered to a woman who has endometriosis and who is to undergo a treatment selected from surgery for resection of endometriotic lesions and/or ART treatment. Typically, the combination according to the invention may be administered daily for at least one week (for example for at least 2, 3, 4, 5, 6, 7, or 8 weeks) before the treatment. This administration may be combined with the administration of an oral contraceptive, typically an estrogen-progestagen pill for example based on levonorgestrel and ethinylestradiol, or else a GnRH agonist.

In another embodiment, the combination according to the invention is administered to a woman who has endometriosis and who has undergone a surgical treatment for resection of endometriotic lesions.

Furthermore, the combination according to the invention may be administered with other active substances having a physiological or nutritional effect, such as prebiotics, vitamins, trace-element minerals; powders or extracts of plants, fruits, vegetables, algae or fungi; and the probiotics and more generally with any physiological substance with a nutritional or health aim, preferably as defined in the Decree of 26 Sep. 2016 establishing the list of substances with a nutritional or physiological purpose permitted in food supplements and the conditions for use thereof in France. Examples of such substances are detailed hereunder in the composition section.

In a particular embodiment, the combination according to the invention is administered together with an active ingredient selected from the group consisting of vitamins, in particular vitamin C, vitamin D, vitamin B6, vitamin B12, folic acid and/or 5-methyltetrahydrofolic acid (both belonging to the vitamin B9 group), minerals such as zinc, copper, magnesium, calcium, trace elements such as selenium, or manganese, a probiotic such as the Lactobacillus spp species, for example Lactobacillus acidophilus, Lactobacillus rhamnosus, Lactobacillus plantarum, Lactobacillus casei, Lactobacillus paracasei or the Bifidobacterium spp species, prebiotics such as inulin, inositol or the fructo/galacto-oligosaccharides, gums (guar, acacia etc.), contraceptive agents and combinations thereof.

As an example, the combination according to the invention may be administered with one or more of the following substances: vitamin B6, vitamin B9, zinc, magnesium, selenium, inulin, a fructo- or galacto-oligosaccharide and combinations thereof.

As an illustration, the combination according to the invention is administered in combination with zinc (Zn2+), for example in the form of the bisglycinate salt and a prebiotic such as inulin.

4. Composition and Kit According to the Invention

The invention also relates to a composition comprising the combination of active ingredients according to the invention, namely resveratrol, a curcuma extract and a yeast Saccharomyces cerevisiae. It goes without saying that said composition is intended in particular to be administered to subjects suffering from endometriosis or are susceptible to endometriosis. Said composition is in particular suitable for implementing any one of the uses and methods described in the present application, in particular for the management of a subject suffering from endometriosis or is susceptible to endometriosis.

Resveratrol, curcuma extract and the yeast Saccharomyces cerevisiae are as defined above. Preferably, the composition according to the invention is a composition for oral administration. It may be, in particular, a pharmaceutical, nutraceutical or nutritional food composition preferably intended for administration to humans. It may be in particular a medicinal product, a food supplement, a functional food, a nutraceutical, a nutritional preparation intended for a specific group, etc.

Preferably, the composition according to the invention is a medicinal product or a food supplement, more preferably a food supplement.

The composition according to the invention may be in various forms. It may be a solid, liquid or gel composition. Preferably, the composition according to the invention is a composition in a form suitable for administration by oral route. It may in particular be in the form of pills, tablets, capsules, powders optionally to be dissolved or to be resuspended in a suitable vehicle, paste or chewing gum, sweets to be sucked or to be crunched, solutions, for example oral solutions packaged in ampules, gel, etc.

In certain embodiments, the composition according to the invention comprises, or consists essentially of, resveratrol, a curcuma extract and yeasts Saccharomyces cerevisiae in the following proportions:
from 10 to 50% by weight of resveratrol,
from 10 to 60% by weight of a curcuma extract,
from 1 to 50% by weight of yeasts Saccharomyces cerevisiae,

the percentages being expressed for 100% by weight of the sum of resveratrol, curcuma extract and Saccharomyces cerevisiae present in said composition or for 100% by weight of the composition.

In another embodiment, the composition according to the invention comprises, or consists essentially of, resveratrol, a curcuma extract and yeasts Saccharomyces cerevisiae in the following proportions:
from 20 to 70% by weight of resveratrol,
from 1 to 20% by weight of a curcuma extract,
from 10 to 60% by weight of yeasts Saccharomyces cerevisiae,

the percentages being expressed for 100% by weight of the sum of resveratrol, curcuma extract and yeast Saccharomyces cerevisiae present in said composition or for 100% by weight of the composition. Preferably, the percentages are expressed for 100% by weight of the sum of resveratrol, curcuma extract and yeast Saccharomyces cerevisiae present in said composition.

In another embodiment, the composition according to the invention comprises, or consists essentially of, resveratrol, a curcuma extract and yeasts Saccharomyces cerevisiae in the following proportions:
from 20 to 40% by weight of resveratrol,
from 40 to 60% by weight of a curcuma extract,
from 5 to 30% by weight of yeasts Saccharomyces cerevisiae,

the percentages being expressed for 100% by weight of the sum of resveratrol, curcuma extract and yeast Saccharomyces cerevisiae present in said composition or for 100% by weight of the composition. Preferably, the percentages are expressed for 100% by weight of the sum of resveratrol, curcuma extract and yeast Saccharomyces cerevisiae present in said composition.

In an additional embodiment, the composition according to the invention comprises, or consists essentially of, resveratrol, a curcuma extract and yeasts Saccharomyces cerevisiae in the following proportions:
from 50 to 70% by weight of resveratrol,
from 5 to 20% by weight of a curcuma extract,
from 15 to 40% by weight of yeasts Saccharomyces cerevisiae,

the percentages being expressed for 100% by weight of the sum of resveratrol, curcuma extract and yeast Saccharomyces cerevisiae present in said composition or for 100% by weight of the composition. Preferably, the percentages are expressed for 100% by weight of the sum of resveratrol, curcuma extract and yeast Saccharomyces cerevisiae present in said composition.

In another embodiment, the composition according to the invention comprises, or consists essentially of, resveratrol, a curcuma extract and yeasts Saccharomyces cerevisiae in the following proportions:
from 25 to 35%, e.g. from 30 to 34% by weight of resveratrol,
from 45 to 55%, e.g. from 46 to 50% by weight of a curcuma extract,
from 15 to 25%, e.g. of 18 to 22% by weight of yeasts Saccharomyces cerevisiae,

the percentages being expressed for 100% by weight of the sum of resveratrol, curcuma extract and Saccharomyces cerevisiae present in said composition or for 100% by weight of the composition.

In certain additional or alternative embodiments, the composition according to the invention is characterized by one or more of the following characteristics:
the curcuma extract is an extract comprising at least 15% by weight of curcuminoids, and/or
the curcuma extract comprises curcumin, demethoxycurcumin and/or bisdemethoxycurcumin, and/or
the curcuma extract comprises at least 55% by weight, preferably at least 60% by weight of curcumin relative to the total weight of the curcuminoids present in the extract, and/or
the curcuma extract is encapsulated in a cyclodextrin, preferably a gamma cyclodextrin, and/or
the composition comprises from 1 to 20% by weight, preferably from 1 to 10% by weight of curcuminoids for 100% by weight of the sum of resveratrol, curcuma extract and Saccharomyces cerevisiae or for 100% by weight of the composition, and/or
the composition comprises from 0.5 to 10% by weight of curcumin for 100% by weight of the sum of resveratrol, curcuma extract and Saccharomyces cerevisiae or for 100% by weight of the composition, and/or
the yeast Saccharomyces cerevisiae is a yeast of the boulardii variety and/or
resveratrol is supplied in the form of a resveratrol-enriched plant extract, preferably an extract of Japanese knotweed and/or
resveratrol is supplied in the form of trans-resveratrol.

It goes without saying that the composition according to the invention may comprise one or more additional ingredients for example one or more additives, excipients or additional active substances as defined hereunder. This or these additional ingredients are preferably selected from pharmaceutically, nutraceutically and/or nutritionally acceptable ingredients. They may represent from 1% to 95%, preferably from 1% to 79%, for example from 10% to 50%, or else from 20% to 60%, by weight relative to the total weight of the composition according to the invention.

Thus, the composition according to the invention may comprise:
from 5% to 99% by weight, preferably from 21% to 99% by weight of resveratrol, of yeast Saccharomyces cerevisiae and of curcuma extract, preferably in the aforementioned proportions, and
from 1% to 95% by weight, preferably from 1% to 79% by weight of one or more ingredients selected from additives, excipients and additional pharmaceutically, nutraceutically and/or nutritionally acceptable active ingredients.

As an additional example, the composition according to the invention may comprise:
from 10 to 50% by weight of resveratrol,
from 10 to 60% by weight of a curcuma extract,
from 1 to 50% by weight of yeasts Saccharomyces cerevisiae, and
from 1 to 79% by weight of one or more additional ingredients selected from additives, excipients and additional pharmaceutically, nutraceutically and/or nutritionally acceptable active ingredients.

As another example, the composition according to the invention may comprise:
from 10 to 50% by weight of resveratrol,
from 1 to 20% by weight of curcuminoids in the form of a curcuma extract,
from 1 to 50% by weight of yeasts Saccharomyces cerevisiae, and
from 1 to 88% by weight of one or more additional ingredients selected from additives, excipients and additional pharmaceutically, nutraceutically and/or nutritionally acceptable active ingredients.

The additional ingredients may be of any type and depend on the form and on the therapeutic, nutraceutical, or physiological effects sought for the composition according to the invention. In a particular embodiment, the composition comprises one or more additional pharmaceutical or nutraceutical active ingredients, including one or more physiological ingredients for a nutritional or health purpose, preferably as defined in the Decree of 26 Sep. 2016 establishing the list of ingredients with a nutritional or physiological purpose permitted in food supplements and the conditions for use thereof in France. The one or more additional pharmaceutical or nutraceutical active ingredients preferably represent at most 50% by weight, in particular at most 45, 40, 30, 20, 15 or 10% by weight relative to the total weight of the composition. Typically, they are present in an amount from 0.5% to 30% by weight of the final weight of the composition.

The active pharmaceutical ingredients include in particular NSAIDs, contraceptives in particular progestogens such as desogestrel and levonorgestrel and dienogest estrogens such as ethinylestradiol, GnRH agonists and combinations thereof.

The nutraceutical active ingredients of interest in particular for a nutritional or physiological purpose encompass, without being limited to, vitamins, minerals, trace elements, extracts and powders of plants, algae or fungi, prebiotics, probiotics, amino acids, antioxidants and combinations thereof.

As an example of vitamins, minerals and trace elements, we may mention a vitamin (thiamine (B1), riboflavin (B2), niacin (B3), pantothenic acid (B5), pyridoxine (B6), folic acid (B9) and cyanocobalamin (B12), as well as the vitamins C, A, D, E, K1 and K2, a mineral such as magnesium, calcium, or iron, trace elements such as iodine, iron, copper, zinc, selenium, chromium, molybdenum, boron, manganese, or a mixture thereof.

As an example, the powders or extracts (dry or liquid) of plants, algae or fungi may in particular be selected from the various European regulatory lists known by a person skilled in the art comprising for example: konjac powder/extract, yam powder/extract, beans powder/extract, coffee powder/extract, tea powder/extract, pine bark powder/extract, grape powder/extract, garlic powder/extract, saffron powder/extract, apple powder/extract, reishi powder/extract, spirulina powder/extract, chlorella powder/extract, brown algae powder/extract and any mixture thereof.

As an example, the prebiotic may be a sugar, a sugar alcohol, an oligosaccharide or a more or less branched polysaccharide with a variable degree of polymerization. The prebiotic may for example be selected from inulin, inositol, tagatose, lactulose, alpha-glucan oligosaccharide, the transgalacto-oligosaccharides (TOS), the fructo-oligosaccharides (FOS), the galacto-oligosaccharides (GOS), the xylo-oligosaccharides (XOS) and mixtures thereof.

As an example, amino acids encompass, without being limited to, leucine, citrulline, glutamine, glutamate, cysteine and derivatives thereof, methionine and derivatives thereof, tryptophan and derivatives thereof.

As an example, the antioxidants encompass, without being limited to, superoxide dismutase (SOD), ubiquinol/ubiquinone (Coenzyme Q10), resveratrol, the catechins (catechin, epicatechin and gallate derivatives), flavanols, flavanones or anthocyanins.

Probiotics encompass, without being limited to, the microorganisms selected from the group consisting of the microorganisms of the genus Bacillus, Bacteroides, Bifidobacterium, Enterobacteriaceae, Enterococcus, Faecalibacterium, Fusobacterium, Kluyveromyces, Lactobacillus, Odoribacter, Parabacteroides, Pediococcus, Ruminococcus, Streptococcus or other yeasts.

In a particular embodiment, the composition according to the invention comprises fewer than 20 different strains of microorganisms, preferably fewer than 10, 9, 8, 7, 6, 5, 4, or 3 different strains of microorganisms.

Besides or additionally, the pharmaceutical, nutraceutical or nutritional composition may also comprise one or more prebiotics.

According to one embodiment, the pharmaceutical, nutraceutical or food composition according to the invention comprises a probiotic in the live form as well as one or more prebiotics that may be degraded by the probiotic or the intestinal microbiota. This association of prebiotic(s) and probiotic constitutes a synbiotic.

In a particular embodiment, the composition according to the invention comprises a strain of Lactobacillus in particular Lactobacillus rhamnosus or acidophilus as an additional probiotic. For example, the composition according to the invention comprises a strain of Saccharomyces cerevisiae, preferably of the boulardii variety and a strain of Lactobacillus, preferably Lactobacillus acidophilus.

In another embodiment, the composition is devoid of any probiotic belonging to the genus Lactobacillus, in particular Lactobacillus acidophilus, and/or to the genus Bifidobacterium.

In an additional embodiment, the yeast Saccharomyces cerevisiae is the only probiotic present in the composition according to the invention.

In a particular embodiment, the composition according to the invention comprises an additional nutraceutical or pharmaceutical active ingredient selected from the group consisting of vitamins, in particular vitamin C, vitamin B6, vitamin B12, folic acid and/or 5-methyltetrahydrofolic acid (vitamin B9), a trace element or mineral such as zinc, magnesium, calcium, selenium, manganese, a probiotic such as the Lactobacillus spp species, a ingredient with a nutritional or physiological purpose such as inulin, the fructo/galacto-oligosaccharides, gums (guar, acacia . . . ) or inositol and a combination thereof.

In another particular embodiment, the composition according to the invention comprises an additional nutraceutical or pharmaceutical active ingredient selected from the group consisting of vitamins, in particular vitamin C, vitamin D, vitamin B6, vitamin B12, folic acid and/or 5-methyltetrahydrofolic acid, and a trace element or mineral such as zinc, magnesium, calcium, selenium or manganese.

As an example, the composition comprises, in addition to the combination according to the invention, one or more of the ingredients selected from the group consisting of vitamin B6, vitamin B9, zinc, magnesium, selenium, inulin, a fructo/galacto-oligosaccharide and combinations thereof.

For example, the composition according to the invention comprises zinc and vitamin B6.

According to another example, the composition according to the invention may comprise zinc and inulin.

In a particular embodiment, the composition according to the invention is in the form of dosage units comprising:
from 10 to 300 mg, preferably from 25 to 250 mg of resveratrol,
from 10 to 300 mg, preferably from 25 to 250 mg of a curcuma extract, and
from 1×10⁷ CFU to 1×10¹¹ CFU, preferably 1×10⁸ CFU to 1×10⁹ CFU of yeast Saccharomyces cerevisiae, preferably of the boulardii variety.

In another particular embodiment, the composition according to the invention is in the form of dosage units comprising:
from 25 to 100 mg, preferably from 30 to 70 mg of resveratrol,
from 1 to 300 mg, preferably from 5 to 25 mg of a curcuma extract, and
from 1×10⁸ CFU to 1×10⁹ CFU, preferably from 2×10⁸ CFU to 6×10⁸ CFU of yeast Saccharomyces cerevisiae, preferably of the boulardii variety.

The dosage units may be for example tablets and capsules.

The formulation of a pharmaceutical, nutraceutical or food composition according to the present invention may vary depending on the route of administration and the dosage for which the composition is intended to be used. The pharmaceutical, nutraceutical or food composition according to the invention may in particular be in solid, semisolid or liquid form. After formulation with at least one physiologically acceptable vehicle or excipient, a composition according to the invention may be in any form suitable for administration to a mammal, in particular humans, for example in the form of tablets, pastilles, sugar-coated pills, soft capsules, hard capsules, pills, granules, powder, suspensions, emulsions, syrups, ointments, ampules of liquids, bottles provided with a dropper and other similar forms of liquid preparations or powders, or suppositories.

A person skilled in the art is able to select the vehicles and excipients that are the most suitable for preparing a given type of formulation.

In a particular embodiment, the physiologically acceptable vehicle may be a solid and the composition according to the invention may be in the form of a powder or a tablet. The physiologically acceptable vehicle may alternatively be a liquid, and the composition according to the invention is in the form of a solution. Liquid vehicles are used in the preparation of solutions, solvents, dispersion media, suspensions, emulsions, syrups, elixirs and pressurized compositions. Suitable liquid or gel-based carriers comprise, but are not limited to: water and physiological saline solutions; emulsions or suspensions, including saline solutions and buffered media, urea; nonaqueous solvents such as alcohols (for example, ethanol) or vegetable oils or seed oils such as olive oil. Liquid compositions, including for example emulsions, microemulsions, solutions, suspensions, syrups, or elixirs may be formulated in particular in the presence of solvents, solubilizers, emulsifiers, oils, fatty acids, and/or other additives for example such as suspending agents, preservatives, sweeteners, natural or synthetic flavorings, stabilizers and/or thickeners and/or food-grade coloring matter. Emulsions may contain emulsifiers such as lecithin, sorbitan monooleate or acacia. Well-known thickeners may also be added to the compositions such as maize starch, natural or synthetic gums, resins, methylcellulose, carboxymethylcellulose sodium, guar gum, xanthan gum and the like.

The excipients and vehicles are selected so as not to affect significantly the viability of the yeast Saccharomyces cerevisiae and other probiotics optionally present in the composition according to the invention.

In particular, the composition according to the invention may further comprise:
anticaking agents such as magnesium carbonate, calcium carbonate, silicon dioxide, stearic acid, a fatty acid salt for example magnesium stearate, magnesium palmitate, magnesium oleate or magnesium myristate; and/or
one or more viscosity agents or surfactants, in particular starch, cellulose ethers such as hydroxypropylmethylcellulose or gum arabic; and/or
one or more binders, in particular fish oil or any vegetable oil such as for example sunflower oil, soybean oil, olive oil or chia oil; and/or
coating agents such as beeswax, carnauba wax or the monoglycerides of food fatty acids; and/or
emulsifiers such as lecithin, cyclodextrins or ester gum; and/or
one or more diluents such as cellulose or maltodextrin
one or more non-nutritive sweeteners, in particular sorbitol, sucralose, aspartame, neotame, maltitol, xylitol, acesulfame-K, aspartame, saccharin, glycyrrhizic acid or a stevia extract; and/or
one or more natural or artificial flavorings, in pulverulent or liquid form; and/or
one or more acidity correctants, in particular salt, citric acid, malic acid or tartaric acid; and/or
one or more colorants, in particular anthocyanins, beta carotene, beet extract, lycopene or caramel extract.

In certain embodiments, a pharmaceutical, nutraceutical or food composition according to the present invention is formulated for immediate release of the active ingredient or ingredients. Alternatively, a pharmaceutical composition may be formulated for prolonged or targeted release of the active ingredient(s) or for protection of the active ingredient(s), for example against acidity and gastric enzymes. It is thus possible to use coatings resistant to the pH and/or to the action of the gastric enzymes, coatings sensitive to the pH and/or to the enzymatic effects, or bioadhesive coatings that adhere to the walls of the stomach or intestine, or encapsulation systems.

Preferably, the composition is in a gastro-resistant oral form allowing the yeasts contained in the composition according to the invention to withstand the gastric juice of the stomach and be released in the intestine. An enteric coating may be resistant to acid pH (as in the stomach) and may dissolve at a basic pH (for example, in the intestine). The material that may be used in enteric coatings comprises, for example, alginic acid, cellulose acetate phthalate, waxes, shellac, fatty acids (for example stearic acid or palmitic acid) or chitosan, not exclusively.

In one embodiment, the gastro-resistant and entero-soluble coating is designed for maintaining the stability of the active ingredients in the stomach. The enteric coating is designed for being maintained in acidic conditions of the stomach and to degrade in nonacidic conditions, thus releasing the pharmaceutical, nutraceutical or food composition in the intestine.

The enteric coatings may be used for i) preventing the gastric juice reacting with or destroying the active ingredient, ii) preventing dilution of the active ingredient before it reaches the intestine, iii) ensuring that the active ingredient is only released after passage of the preparation through the stomach, and iv) preventing the live yeasts contained in the composition according to the invention being altered or killed by the acid pH of the stomach.

According to one embodiment, the composition may further comprise maltodextrin and/or magnesium stearate.

The composition according to the invention may be prepared by any method known by a person skilled in the art, for example by mixing resveratrol, curcuma extract and the yeast Saccharomyces cerevisiae with one or more nutritionally, nutraceutically or pharmaceutically acceptable excipients or additives.

In a particular embodiment, the composition according to the invention is in the form of dosage units comprising:
from 25 to 100 mg, preferably from 30 to 70 mg of resveratrol,
from 1 to 300 mg, preferably from 5 to 25 mg of a curcuma extract, and
from 1×10⁸ CFU to 1×10⁹ CFU, preferably from 2×10⁸ CFU to 6×10⁸ CFU of yeast Saccharomyces cerevisiae, preferably of the boulardii variety,
a prebiotic, preferably inulin, and
zinc, preferably in the form of zinc bisglycinate.

The composition according to the invention may further comprise one or more excipients selected from the group consisting of a diluent, an anticaking agent, an emulsifier, a viscosity agent and combinations thereof.

In particular, the composition according to the invention may comprise gamma-cyclodextrin, maltodextrin and a fatty acid salt, for example magnesium stearate or a mixture of magnesium salts of stearic, oleic, palmitic and myristic acids

Preferably, the dosage units according to the invention are in the form of a powder. This powder is typically contained in a capsule, preferably a capsule based on hypromellose (HPMC) and calcium carbonate.

As an illustration, the composition according to the invention is in the form of dosage units comprising:
from 25 to 100 mg, preferably from 30 mg to 70 mg of resveratrol, preferably trans-resveratrol,
from 5 to 25 mg of curcuminoids in the form of a curcuma extract,
from 1×10⁸ CFU to 1×10¹⁰ CFU, preferably from 2×10⁸ CFU to 6×10⁸ CFU, more preferably from 3×10⁸ CFU to 5×10⁸ CFU of yeast Saccharomyces cerevisiae of the boulardii variety, for example the strain CNCM 1-3799 or the strain CNCM 1-745
from 10 mg to 100 mg, preferably from 25 mg to 50 mg of inulin, for example supplied in the form of an extract of chicory root
from 1 mg to 15 mg of zinc (Zn2+) preferably from 2 mg to 8 mg of zinc, for example in the form of zinc bisglycinate.

The dosage unit may be contained in a capsule, preferably vegetable, based on HPMC or pullulan.

The dosage unit may comprise, as excipients, gamma-cyclodextrin, maltodextrin and a magnesium salt of fatty acids, for example a magnesium salt of stearic, oleic, palmitic, myristic acid and a mixture thereof.

Gamma-cyclodextrin is typically present for improving the bioavailability of the curcuma extract. Gamma-cyclodextrin may be present in an amount from 20 to 120 mg per dosage unit. Maltodextrin may be present in an amount from 5 to 50 mg per dosage unit. The magnesium salt of fatty acid may be present in an amount from 0.1 mg to 10 mg per dosage unit.

The invention relates additionally to a nutraceutical or pharmaceutical kit, for example a food supplement kit comprising several compositions, preferably intended for oral administration, incorporating the combination according to the invention.

In a particular embodiment, said kit comprises:
a composition comprising the yeast Saccharomyces cerevisiae,
a composition comprising resveratrol, and
a composition comprising the curcuma extract.

In a particular embodiment, the kit comprises:
a first composition comprising the yeast Saccharomyces cerevisiae, for example associated with one or more excipients in a capsule,
a second composition comprising resveratrol, and curcuma extract, for example in the form of a tablet or a capsule.

The kit typically comprises several dosage units of each composition. It may also comprise instructions for administration of the kit.

In a particular embodiment, the first composition may be in the form of dosage units comprising from 10 to 300 mg, preferably from 25 to 250 mg of resveratrol and from 1 to 300 mg, preferably from 10 to 300 mg, from 5 to 25 mg or from 25 to 250 mg of a curcuma extract, and the second composition may be in the form of dosage units comprising from 1×10⁷ CFU to 1×10¹¹ CFU, preferably 1×10⁸ CFU to 1×10⁹ CFU of yeast Saccharomyces cerevisiae, preferably of the boulardii variety. The dosage units may be for example tablets and capsules. The characteristics of the compositions present in this kit are similar to those of the composition according to the invention, in particular in terms of the relative weight ratios between yeast, resveratrol and curcuma extract, and doses.

Each composition present in the kit may comprise one or more additional ingredients selected from additives, excipients and additional pharmaceutically, nutraceutically and/or nutritionally acceptable active ingredients as described above.

The invention also relates to a method of treatment of a subject suffering from endometriosis or susceptible to endometriosis comprising the administration of an effective dose of resveratrol, a curcuma extract and yeast Saccharomyces cerevisiae to said subject. The implementation of such a method and the expected effects are those described for the uses according to the invention.

The invention will be better understood on reading the examples given hereunder, which are only illustrative and are not limiting.

### EXAMPLES

### Example 1: Evaluation of the Effect of the Combination According to the Invention According to the Invention on a Murine Model of Endometriosis

The objective of this study was to test in vivo the effect of a combination according to the invention comprising a curcuma extract, resveratrol and a yeast S. cerevisiae var. boulardii on the growth of endometriotic lesions and the immune and inflammatory state in a murine model of endometriosis.

Material and Methods

Female Balb/CJrj mice aged 6 weeks were purchased from the supplier Janvier Labs (Route du Genest, 53940 Le Genest-Saint-Isle). The mice are kept in a conventional animal house, with automated day/night cycles of 12 h, drink (tap water) and food (Teklad, Envigo, Gannat, France, #T.2016MI.12) ad libitum. The endometriotic mouse model by implantation (suture) of syngeneic intraperitoneal uterine horn is a validated model that has been used several times and published [Jones et al., 2018; Ruiz et al., 2016]. The implantation of uterine horns intraperitoneally is carried out under isoflurane anesthesia (3.5%, under oxygen 2.5 l/min) and post-operative analgesia (Buprëcare, 10 μg/kg, sc). A laparotomy is carried out and a fragment is fixed on each side of the opening, on the inside wall of the peritoneum, by two suture points made with absorbable suture 5/0.

The molecules and products used for the treatments are: Zinc-Bisglycinate Chelate (ALBION Laboratories, Clearfield, USA, #03506), Gum Acacia Seygal (Nexira Food, Rouen, France, #FibergumB), Trans-resveratrol (Active Inside, Beychac et Caillau, France, #PR-0018), curcuma extract (Wacker, Eddyville, Iowa, USA, #60072151), Saccharomyces Boulardii (LHC Lesaffre Bio Springer, Maison Alfort, France, #VI1700368), Lactobacillus Acidophilus (Danisco US, Madison, Wis., USA, #MSAMPHRUDOPH).

The groups of mice are as follows:
Control Group (Sham) (n=10): Mice with sham surgery but without implant and without treatment (Sham);
Group M (control) (n=10): mice operated on, treated with a composition comprising the Vehicle (Zinc-Bisglycinate, Gum Acacia-Seygal, Trans-resveratrol and Curcuma extract) and therefore without probiotic;
Group M1 (Invention) (n=20): mice operated on, treated with a composition comprising the Vehicle and 0.36 mg/d of Saccharomyces Boulardii;
Group M2 (Invention) (n=20): mice operated on, treated with a composition comprising the Vehicle, 0.36 mg/d of Saccharomyces Boulardii and 0.18 mg/d of Lactobacillus Acidophilus); The compositions were suspended in aqueous phase and were administered to the mice per os by stomach tube (1 gavage per day). The solutions are prepared extemporaneously and administration is carried out in the morning. For each group, the various solutions are administered over 12 consecutive weeks. At S3 (three weeks) and S12 (12 weeks), the animals will be sacrificed (n=5 per group Sham and M and n=10 per group M1 and M2 at S3 and S12). The blood, the spleen and the lesions are collected. The size and the weight of the lesions will be evaluated and the following analyses will be carried out.

The following tables present the characteristics of the test:

TABLE 1
Distribution of the animals (n = 10 per group)

| Groups | Treatments |
|---|---|
| Sham − Sham surgery | / |
| Model induced + M | Resveratrol + Curcuma extract |
| Model induced + M1 | Resveratrol + Curcuma extract + |
| | Saccharomyces boulardii |
| Model induced + M2 | Resveratrol + Curcuma extract + |
| | Saccharomyces boulardii + |
| | Lactobacillus acidophilus |

TABLE 2
Amount of raw materials administered to the mice by stomach tube

| | Amounts | Amounts supplied by |
|---|---|---|
| Ingredients | supplied/d (mg) | stomach tube (mg) |
| Saccharomyces boulardii - | 0.36 | 0.36 |
| LYNSIDE PRO ® - | | |
| CNCM No. I-3799 (group | | |
| M1 and M2) | | |
| Lactobacillus acidophilus | 0.18 | 0.18 |
| NCFM (group M2) | | |
| Polygonum extract with | 0.6 | 0.6 |
| 98% Resveratrol | | |
| Curcuma extract | 0.9 | 0.9 |

The change in volume of the lesions is monitored by ultrasonography.

With high-resolution ultrasonography (40 m) it is possible to monitor the changes in the size of the implants of uterine horns accurately and noninvasively and quickly evaluate the progression of endometriosis. This procedure is carried out at PIPA (Small Animal Imaging Platform of Cochin Institute) at 51, S3, S8, S11 after implantation of the horn fragments.

After euthanasia, samples are taken (at S3 and S12) in order to measure various parameters.
Histology of the lesions by Hemalaun/Eosin (HE) staining

The 5 μm sections of the lesions fixed in paraffin were stained by the conventional HE technique.
Immune state in the spleens by FACS (LT, NK, macrophages)

Specific antibodies were used in order to determine the proportions of T lymphocytes CD4 and CD8, Natural Killer and splenic macrophages:

MIX 1: B220 APC, CD69 PE, CD8 BV605, CD40 PerCP/Cy5.5, MHCII FITC, CD4 BV510-MIX 2: CD206 AF647, CD86 BV510, Ly6C PE VIOL.770, CD1 lb APC-Cy7 and F4/86 BV711. The FACS used is an LSR Fortessa BD and the analysis software BD FACS Diva 6.2 software.
Assays in the sera:

AOPP measured by spectrophotometry on the plate reader in a 96-well plate according to the published technique (Kidney Int. 1996 May; 49(5): 1304-13. Advanced oxidation protein products as a novel marker of oxidative stress in uremia. Witko-Sarsat V et al.)

IL6 and TNFa are measured with ELISA kits (Thermofisher Scientific, Waltham, Mass. USA #KMC0061 and BMS607-3 respectively) as Zonulin (Clinisciences, Nanterre, France, #MBS748504-96).
Investigation of the lesions by histology and qRT-PCR

The total RNA was extracted from the mouse tissues (lesions of the endometriosis type) using the reagent Trizol (Invitrogen, Carlsbad, USA), according to the manufacturer's instructions. The cDNA was synthesized using the synthesis kit Maxima H Minus cDNA Synthesis Master Mix kit (Thermo Fisher Scientific). The qPCR was carried out using the kit SensiFAST SYBR No-ROX (Bioline). The transcription levels of the selected genes were quantified in a Light Cycler 480 (Roche, San Francisco, Calif.) and normalized for the reference genes (Actinb and Tbp) and analyzed using the LightCycler dedicated software.

Statistical Analysis of the Various Results

Comparative statistical analysis of the various groups of mice is carried out by 1-factor ANOVA followed by a Kruskal-Wallis test. The comparison S3-S12 by ANOVA followed by a Bonferroni test.

Results

1) Weight and Volume of the Endometriotic Lesions

The weights and volumes of the lesions are uniform at implantation for the three groups of mice M, M1 and M2 (M, n=10; M1, n=20; M2, n=20).

After 12 weeks of treatment, it is noted that the volume and the weight of the lesions are significantly lower for the mice that were treated with a composition according to the invention (M1 or M2) than for the mice treated with the control composition M (p<000.1).

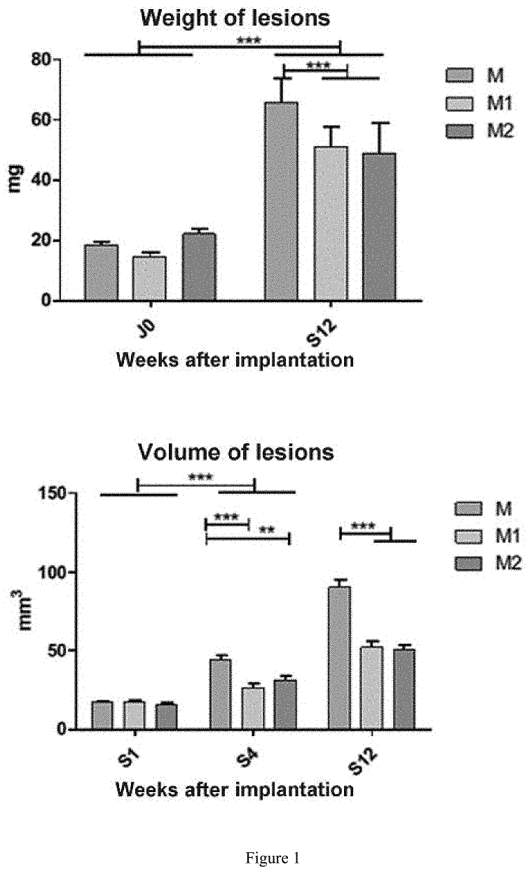

Thus, the combination according to the invention combining resveratrol, a curcuma extract and the yeast Saccharomyces cerevisiae boulardii acts synergistically on the development of the endometriotic lesions. (FIG. 1).

2) Histology of the Lesions Sampled at Sacrifice

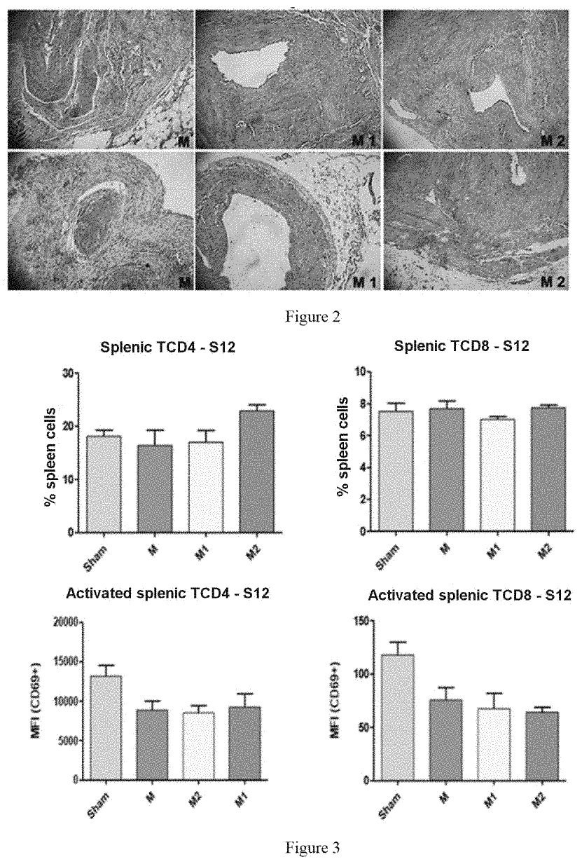

Histologically, a decrease in activity of the lesions is observed in groups M1 and M2 relative to the control M, after 3 and 12 weeks of treatment (FIG. 2): in fact, the glandular appearance, a marker of the endometriotic lesions, is clearly attenuated in groups M1 and M2 relative to group M.

3) Splenic Immune State in the Various Groups, at S12

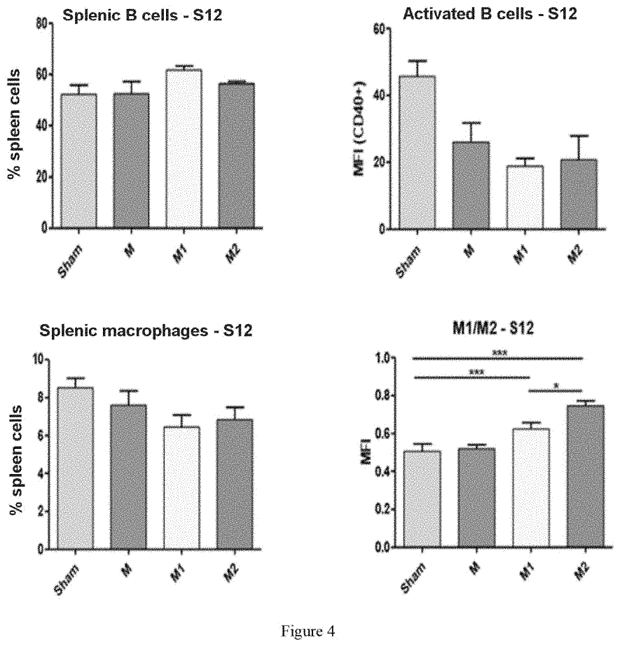

The immune state is altered in all the groups compared to the sham surgery group without lesion implantation (SHAM) (FIGS. 3 and 4).

The percentages of activated TCD4 and TCD8 of the spleen tend to decrease without significance between the groups Sham, M, M1 and the group M2 (Sham, n=3−M, n=6− M1, n=5− M2, n=5). The percentage of activated B cells of the spleen tends to be reduced by formulations M, M1 and M2 (Sham, n=3−M, n=6−M1, n=5−M2, n=5).

It is noted that the ratio M1/M2 (type 1 and 2 macrophages) is significantly increased in the mice in the groups according to the invention (M1 and M2) relative to the control groups (M and Sham). Studies in humans and in mice have demonstrated the key role of the M2 macrophages in the pathogenesis of the establishment and growth of endometriotic lesions. In fact, there was shown to be an increase in the proportion of M2 macrophages in the peritoneal liquid of women with endometriosis, relative to the controls. Moreover, it has also been shown that intraperitoneal transfer of M2 macrophages leads to growth and neovascularization of endometriosis in mice (Bacci et al., 2009). Thus, the results of the present study suggest that the combination according to the invention has an immunomodulating effect on the polarization of the macrophages, which might allow normalization of the immune response in patients suffering from endometriosis.

4) Inflammatory Parameters

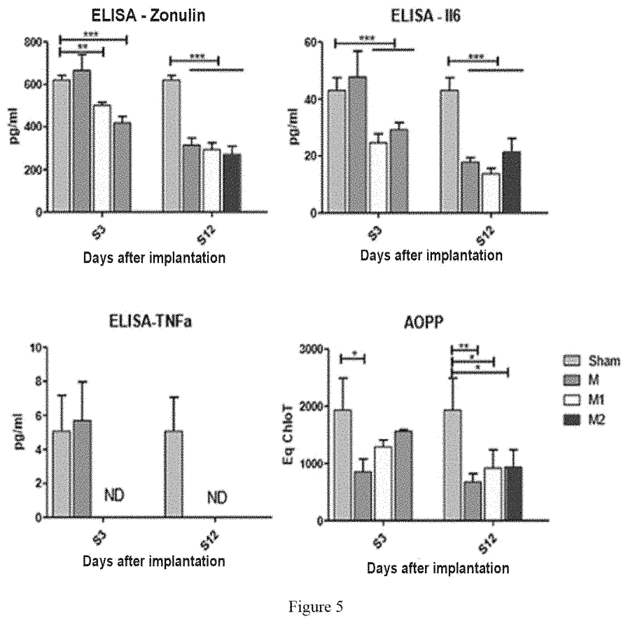

Zonulin, a marker of permeability of the intestinal barrier, and interleukin 6, a marker of inflammation, are very significantly reduced in the groups according to the invention (M1 and M2, respectively), compared to the sham surgery control group (SHAM). TNF-α is also decreased very significantly as it is not detectable in the sera from groups M1 and M2 (FIG. 5).

5) Oxidative Stress

The advanced oxidation protein products (AOPP), marker of the level of oxidation of the serum proteins and of oxidative stress, are reduced significantly in the three supplemented groups (M, M1 and M2) at 12 weeks relative to the sham surgery group (Sham) (FIG. 4).

6) qPCR Results

The preliminary results show a tendency for decrease of the angiogenesis marker (Cd31) for the groups M1 and M2 at S12. S12-S3 comparison by ANOVA analysis followed by a Bonferroni test shows a significant decrease only for the marker Igf1 (pain marker) for group M1.

Regarding sensitivity to pain, it is to be noted that sensitivity to heat was significantly reduced in group M1 relative to groups M0 and M2 at week 4 of treatment according to the “hot plate” test.

### CONCLUSIONS

The above results show that the combination according to the invention has favorable effects both in clinical respects and immune and physiological respects. The combination according to the invention slows the growth of the endometriotic lesions significantly in the murine model. Moreover, the endometriosis is characterized by a disturbed immune function. Now, the combination according to the invention produces immunomodulating effects allowing inflammation to be reduced, as reflected in a lowering of the serum levels of the pro-inflammatory cytokines TNF-α and IL-6, modulation of the ratio of the macrophages M1/M2 and reduction of activation of the B cells. Finally, the combination according to the invention makes it possible to reduce the circulating level of zonulin, a marker of intestinal permeability.

### Example 2: Prospective, Randomized, Double-Blind Study for Evaluating the Effect of a Food Supplement Containing the Combination According to the Invention on the Quality of Life of Women with Painful Endometriosis

Study centers:

COCHIN Hospital—Port Royal, France

Methodology:

This pilot study is a single center, randomized, parallel-arm, double-blind, placebo controlled study.
Products:
the experimental product (called Qolendo hereinafter) is a food supplement comprising the combination according to the invention (dosage unit: resveratrol 50 mg, S. boulardii: 400 million CFU and curcumin 12.7 mg) inulin (34 mg), zinc bisglycinate (20 mg eq., gamma-cyclodextrin (58.5 mg), magnesium stearate (1.5 mg) and maltodextrin (19.5 mg), in the form of a capsule (HPMC, calcium carbonate).
the control product is a placebo capsule with the same characteristics of packaging and appearance, in which the active ingredients have been replaced with maltodextrin, β-carotene (to give an orange color) and magnesium stearate.
Population studied:

80 women suffering from painful endometriosis and meeting the criteria for inclusion and exclusion will be randomized in this study.
Planning of the study:

Recruitment: 10 months, Duration for taking the product: 4 months, Total duration of participation per subject: 5 months, Total duration of the study: 15 months
Intervention:

After randomization at visit V1, the subjects will consume:
the experimental product: Qolendo group

1 capsule of the product Qolendo per day from visit 1 to visit 3 (4 months of taking the product) morning or evening, with a glass of water.

Or
the control group: placebo

1 capsule of placebo per day from visit 1 to visit 3 (4 months of taking the product) morning or evening, with a glass of water.
Objective of the study

Study hypothesis: The hypothesis of the study is that the test product Qolendo improves the quality of life of women with painful endometriosis.

Main objective: The main objective is to evaluate the quality of life for women with endometriosis in the Qolendo group relative to those in the placebo group, using the EHP-5 questionnaire after taking the product for 4 months.

Secondary objectives: The secondary objectives of the study are to evaluate the following parameters, between the 2 groups and the intra-group difference:
1) The quality of life using the EHP-5 questionnaire after taking the product for 2 months,
2) The Clinical Global Impression Scale (CGI) questionnaire after taking the product for 4 months,
3) The pains felt throughout the study,
4) The consumption of analgesics throughout the study,
5) The inflammatory, immune and hormonal state after taking the product for 4 months,
6) The permeability of the intestinal epithelium after taking the product for 4 months,
7) The acceptability of the product in the Qolendo group after taking the product for 2 and 4 months.

Exploratory objectives: Pelvic ultrasonography carried out for a subgroup of women with deep endometriosis and/or with the presence of adenomyosis, Evaluation of the quality of life using the EHP-30 questionnaire, completed on a voluntary basis: this questionnaire will be completed at the randomization visit (V1), and at visit V3, after taking the product for 4 months.

Safety objectives: The safety objectives of the study are to evaluate the incidence of adverse events (AEs) and the general state of health by means of the vital signs, body weight and biological parameters (measured at 4 months).
Judgment criteria

Main judgment criterion: The main criterion of this study is the change in the quality of life, at 4 months relative to the initial visit (V1), evaluated by the EHP-5 questionnaire, overall score, expressed in arbitrary units (a.u., score), comparing the Qolendo group versus the placebo group.

Secondary judgment criteria: The secondary criteria of this study, listed below, make it possible to compare the Qolendo group and the placebo group:

1) The change in the quality of life at 2 months relative to the initial visit, evaluated by the EHP-5 questionnaire, expressed in a.u.,

2) The change in the Clinical Global Impression Scale questionnaire at 4 months relative to the initial visit. This questionnaire is made up of 3 sub-scales measuring:
a. The severity of the disease (score ranging from 1 normal to 7 the most severe cases),
b. The overall improvement or the change (score ranging from 1 strong improvement to 7 strong deterioration),
c. The therapeutic response (score ranging from 0 marked improvement without adverse events to 4 no change or deterioration and unsatisfactory benefit/risk balance).

3) The variation of the pain, throughout the study relative to the initial value, defined on visual analog scales (VAS) graduated from 0 to 10 evaluating:

Pain (overall evaluation), Dysmenorrhea, Dyspareunia, Noncyclic chronic pelvic pain, Digestive pain, Dyschesia, Pain when urinating. The dates of menstruation will be collected in order to distinguish catamenial pains.

4) The quantitative consumption of analgesics (in g/day) throughout the study, relative to the initial value,

5) The inflammatory state at 4 months compared to the initial value evaluated by the following parameters: TNF-α, IL1, IL6, CRP-us, AOPP,

6) The immune state at 4 months compared to the initial value evaluated by the following parameters: IL4, IL10, IL12, INFγ, NK cells,

7) The hormonal state at 4 months compared to the initial value evaluated by estradiol (E2), progesterone and the antimüllerian hormone (AMH),

8) The permeability of the intestinal epithelium at 4 months compared to the initial value evaluated by blood zonulin assay,

9) The acceptability of the product in the Qolendo group after 2 and 4 months of taking the product.

Safety Criteria

The safety criteria of this study, measured at the initial visit and at 4 months are:

1) Body weight (kg),

2) The hemodynamic parameters: Heart Rate (HR, bpm), Systolic Blood Pressure (SBP, mmHg) and Diastolic Blood Pressure (DBP, mmHg),

3) The following biological parameters:
Complete blood count,
Hepatic function by determining the following parameters: ASAT, ALAT and GGT,
Renal function by creatinine assay.

4) Number of adverse events per subject that appeared throughout the study;

5) Proportion of subjects presenting at least one adverse event throughout the study.

Exploratory Criteria:

The exploratory criteria of the study relate to the changes between initial values and those measured at 4 months:
Parameters obtained by pelvic ultrasonography of patients with deep endometriosis and/or presence of adenomyosis,
The EHP-30 questionnaire.

1-17. (canceled)

18. A method for treating a subject suffering from, or susceptible of suffering from, endometriosis comprising the administration of an effective dose of resveratrol, a curcuma extract and a Saccharomyces cerevisiae species to said subject.

19. The method according to claim 18, said method is for delaying the onset of endometriotic lesions, slowing down the development of endometriotic lesions or reducing endometriotic lesions in the subject.

20. The method according to claim 18, wherein said method is for treating or delaying the onset of one or more symptoms or disorders associated with endometriosis which are selected from the group consisting of hypofertility, infertility, pelvic pain, dysmenorrhea, dyspareunia, catamenial pains, urinary pain, intestinal pain, urinary and intestinal disorders, metrorrhagia, proctorrhagia, chronic peritoneal inflammation, intestinal hyperpermeability, chronic fatigue and combinations thereof.

21. The method according to claim 18, said method normalizing intestinal permeability, modulating the immune state, maintaining or improving fertility and/or decreasing oxidative stress in a subject suffering from endometriosis.

22. The method according to claim 18, wherein said Saccharomyces cerevisiae is a Saccharomyces cerevisiae of the variety boulardii.

23. The method according to claim 18, wherein the Saccharomyces cerevisiae species resveratrol and the curcuma extract are contained in a single pharmaceutical, nutraceutical or food composition or are distributed in several separate pharmaceutical, nutraceutical or nutritional compositions which are administered simultaneously or sequentially.

24. The method according to claim 18, wherein the subject suffering from endometriosis has undergone or is to undergo a surgical treatment of resection of the endometriotic lesions, a medicinal protocol of medically assisted reproduction, in vitro fertilization and/or is under contraceptive treatment.

25. The method according to claim 18, wherein the Saccharomyces cerevisiae species, resveratrol and the curcuma extract are included in a food supplement, optionally with one or more ingredients with a physiological or nutritional purpose selected from vitamins, trace elements, minerals, prebiotics, probiotics, antioxidants, amino acids and combinations thereof.

26. The method according to claim 25, wherein said one or more ingredients with a physiological or nutritional purpose are selected from folic acid, 5-methyltetrahydrofolic acid, vitamin B6, vitamin B12, zinc, magnesium, calcium, selenium, manganese, inulin, the fructo/galacto-oligosaccharides, acacia gum, guar gum, inositol and combinations thereof.

27. A pharmaceutical, nutraceutical or food composition or food supplement comprising a combination a Saccharomyces cerevisiae species, resveratrol and a curcuma extract.

28. The pharmaceutical, nutraceutical or food composition or food supplement of claim 27, which comprises:
from 1 to 50% by weight of Saccharomyces cerevisiae,
from 10 to 50% by weight of resveratrol,
from 1 to 50% by weight of curcuma extract,
for 100% by weight of the total sum of resveratrol, curcumin and Saccharomyces cerevisiae or for 100% of the total weight of the composition.

29. The composition according to claim 28, said composition further comprising:
one or more prebiotics; and/or
vitamins, minerals and/or trace elements; and/or
powders or extracts of plants, fruits, vegetables, algae or fungi; and/or
one or more probiotics.

30. The composition according to claim 29, wherein said one or more probiotic is not a bacterium of the genus Lactobacillus.

31. The composition according to claim 27, said composition being in the form of dosage units, tablets, or capsules, each dosage unit comprising:
from 10 mg to 300 mg of resveratrol,
from 5 mg to 25 mg of a curcuma extract, and
from 1×10⁷ CFU to 1×10¹¹ CFU Saccharomyces cerevisiae yeast of variety boulardii.

32. The composition according to claim 27, said composition being in the form of dosage units, tablets, or capsules, each dosage unit comprising:
from 25 mg to 250 mg of resveratrol,
from 5 mg to 25 mg of a curcuma extract, and
from 1×10⁸ CFU to 1×10¹⁰ CFU Saccharomyces cerevisiae yeast of variety boulardii.

33. The composition according to claim 31, said composition comprising inulin and zinc.

34. The composition according to claim 31, wherein said composition comprises dosage units comprising:
from 30 mg to 70 mg of resveratrol or trans-resveratrol,
from 5 mg to 25 mg of curcuminoids in the form of a curcuma extract associated with a cyclodextrin,
from 1×10⁸ CFU to 1×10¹⁰ CFU Saccharomyces cerevisiae of the boulardii variety,
optionally from 10 mg to 100 mg inulin, and
optionally from 1 mg to 15 mg of zinc (Zn²⁺).

35. A pharmaceutical, nutraceutical or nutritional kit comprising the combination of two compositions:
a first composition comprising a Saccharomyces cerevisiae species in an amount from 1×10⁷ CFU to 1×10¹¹ CFU; and
a second composition comprising resveratrol in an amount from 10 mg to 300 mg and a curcuma extract in an amount from 1 mg to 300 mg.

36. The kit according to claim 35, wherein:
the yeast Saccharomyces cerevisiae is a Saccharomyces cerevisiae of the variety boulardii,
the resveratrol extract is supplied in the form of a plant extract enriched with trans-resveratrol,
the curcuma extract comprises at least 15% by weight of curcuminoids,
the curcuma extract comprises curcumin, demethoxycurcumin and bisdemethoxycurcumin,
the curcuma extract is encapsulated in a cyclodextrin, and
inulin is supplied in the form of an extract of chicory root.

37. A method for treating a subject suffering from endometriosis comprising the administration of the composition of claim 31 to said subject.
